(19) United States
(12) Patent Application Publication
(10) Pub. No.: US 2025/0122568 A1
(43) Pub. Date: Apr. 17, 2025

(54) METHODS AND MATERIALS FOR ASSESSING AND TREATING ORAL LICHEN PLANUS

(71) Applicant: Mayo Foundation for Medical Education and Research, Rochester, MN (US)

(72) Inventors: Julia S. Lehman, Rochester, MN (US); Fangyi Xie, Rochester, MN (US); Surendra Dasari, Rochester, MN (US)

(21) Appl. No.: 18/692,467

(22) PCT Filed: Nov. 9, 2022
(86) PCT No.: PCT/US2022/049402

§ 371 Date: Mar. 15, 2024

Related U.S. Application Data
(60) Provisional application No. 63/277,425, filed on Nov. 9, 2021.

(51) Int. Cl. C12Q 1/6883

(52) U.S. Cl. CPC C12Q 1/6883; C12Q 2600/156

(57) ABSTRACT

This document provides methods and materials for assessing and/or treating OLP. For example, methods and materials for using the presence of cells associated with OLP that have a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or combinations thereof to identify the OLP as being an OLP that is likely to proceed to OSCC are provided.

### CROSS-REFERENCE TO RELATED APPLICATIONS

This application claims priority from U.S. Provisional Application Ser. No. 63/277,425, filed Nov. 9, 2021. The disclosure of the prior application is considered part of (and is incorporated by reference in) the disclosure of this application.

### TECHNICAL FIELD

This document relates to methods and materials involved in assessing and/or treating a mammal having oral lichen planus (OLP) that is likely to proceed to oral squamous cell carcinoma (OSCC) or that is likely to follow a benign course of the disorder. For example, this document provides methods and materials involved in identifying and treating a mammal (e.g., a human) with OLP that is likely to proceed to OSCC or that is likely to follow a benign course of the disorder.

### BACKGROUND

Patients with OLP typically require regular monitoring, because they may be at risk of developing mouth cancer in the affected areas. For example, patients with OLP are at increased risk of developing OSCC. To date, there is no reliable predictor to identify patients at higher risk to develop this complication, and frequently, the cancer is already invasive when it is detected. Further, the carcinogenic mechanism of OLP remains obscure. In addition, in patients with severe and diffuse OLP, it can be difficult to know when any regions have transformed to OSCC.

### SUMMARY

This document is based, at least in part, on the discovery that OLP containing cells having a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or combinations thereof can be identified as being an OLP that is likely to proceed to OSCC. In some cases, OLP containing cells that lack a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and a mutated version of a nucleic acid encoding a LRP2 polypeptide can be identified as being an OLP that is likely to follow a benign course of the disorder. For example, mammals (e.g., humans) having OLP that is likely to proceed to OSCC can be distinguished from mammals (e.g., humans) having OLP likely to follow a benign course of the disorder based, at least in part, on the presence of cells having a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or a combination thereof.

Distinguishing between mammals (e.g., humans) having OLP that is likely to proceed to OSCC and mammals (e.g., humans) having OLP likely to follow a benign course of the disorder based, at least in part, on identifying cells of an OLP as having or not having a mutated version of nucleic acid encoding a TP53 polypeptide, a mutated version of nucleic acid encoding a CELSR1 polypeptide, a mutated version of nucleic acid encoding a CASP8 polypeptide, and a mutated version of nucleic acid encoding a KMT2D polypeptide, and optionally also lacks a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or a combination thereof as described herein can allow clinicians and patients to proceed with proper clinical surveillance (e.g., frequency of clinical follow-ups and/or need for surveillance biopsies) and treatment options.

This document also provides methods and materials involved in treating mammals (e.g., humans) identified as having or not having OLP that is likely to proceed to OSCC. For example, this document provides methods and materials for administering topical and/or systemic immunosuppressive or immunomodulatory medications (e.g., corticosteroids, dapsone, azathioprine, tacrolimus, mycophenolate mofetil, or biologic agents) based, at least in part, on OSCC risk. As described herein, mammals having OLP that is likely to progress to OSCC can be administered tailored therapies to achieve disease control while minimizing immunosuppression. Having the ability to use more aggressive systemic agents (e.g., azathioprine, tacrolimus, mycophenolate mofetil, or biologic agents) to achieve better disease control can allow clinicians and patients to proceed with treatment options that mitigate the risk of OSCC. In some cases, mammals (e.g., humans) having OLP that is likely to proceed to OSCC can undergo more regular surveillance (e.g., examination and/or biopsy of involved tissue on a more frequent basis) to detect changes of possible OSCC development early in its course.

This document also is based, at least in part, on the discovery that OLP containing cells having elevated expression of CA1, elevated expression of TNNT3, elevated expression of SYNM, elevated expression of MB, elevated expression of MYH4, elevated expression of ST13P4, elevated expression ofMYLPF, elevated expression of MYH1, elevated expression of TNNC2, elevated expression of SRPRB, or combinations thereof can be identified as being a OLP (e.g., a transforming OLP) that is likely to proceed to OSCC. In some cases, OLP containing cells that lack elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, and elevated expression of SRPRB can be identified as being an OLP that is likely to follow a benign course of the disorder. For example, mammals (e.g., humans) having transforming OLP (which is more likely to proceed to OSCC) can be distinguished from mammals (e.g., humans) having indolent OLP (which is more likely to follow a benign course of the disorder) based, at least in part, on the presence of cells having elevated expression of a CA1 polypeptide or mRNA, elevated expression of a TNNT3 polypeptide or mRNA, elevated expression of a SYNM polypeptide or mRNA, elevated expression of a MB polypeptide or mRNA, elevated expression of a MYH4 polypeptide or mRNA, elevated expression of a ST13P4 mRNA, elevated expression of a MYLPF polypeptide or mRNA, elevated expression of a MYH1 polypeptide or mRNA, elevated expression of a TNNC2 polypeptide or mRNA, elevated expression of a SRPRB polypeptide or mRNA, or a combination thereof.

Distinguishing between mammals (e.g., humans) having transient OLP that is likely to proceed to OSCC and mammals (e.g., humans) having indolent OLP likely to follow a benign course of the disorder based, at least in part, on identifying cells of an OLP as having or not having elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, elevated expression of SRPRB, or a combination thereof as described herein can allow clinicians and patients to proceed with proper clinical surveillance (e.g., frequency of clinical follow-ups and/or need for surveillance biopsies) and treatment options.

This document also provides methods and materials involved in treating mammals (e.g., humans) identified as having or not having transforming OLP. For example, this document provides methods and materials for administering topical and/or systemic immunosuppressive or immunomodulatory medications (e.g., corticosteroids, dapsone, azathioprine, tacrolimus, mycophenolate mofetil, or biologic agents) based, at least in part, on OSCC risk. As described herein, mammals having transforming OLP can be administered tailored therapies to achieve disease control while minimizing immunosuppression. Having the ability to use more aggressive systemic agents (e.g., azathioprine, tacrolimus, mycophenolate mofetil, or biologic agents) to achieve better disease control can allow clinicians and patients to proceed with treatment options that mitigate the risk of OSCC. In some cases, mammals (e.g., humans) having transforming OLP (which is more likely than indolent OLP to proceed to OSCC) can undergo more regular surveillance (e.g., examination and/or biopsy of involved tissue on a more frequent basis) to detect changes of possible OSCC development early in its course.

In general, one aspect of this document features a method for identifying a mammal as having OLP that is likely to progress to OSCC. The method comprises (or consists essentially of, or consists of) (a) determining that a cell of the OLP comprises a mutant version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, or a mutated version of a nucleic acid encoding a LRP2 polypeptide, and (b) classifying the mammal as having the OLP. The mammal can be a human. The cell can be within a cytology brushing sample. The cell can be a squamous epithelial cell or a stromal cell affected by the OLP. The method can comprise determining that the cell comprises the mutant version of nucleic acid encoding a TP53 polypeptide. The method can comprise determining that the cell comprises the mutant version of nucleic acid encoding a CELSR1 polypeptide. The method can comprise determining that the cell comprises the mutated version of nucleic acid encoding a CASP8 polypeptide. The method can comprise determining that the cell comprises the mutated version of nucleic acid encoding a KMT2D polypeptide. The method can comprise determining that the cell comprises the mutated version of nucleic acid encoding a TENM3 polypeptide. The method can include determining that the cell comprises the mutated version of nucleic acid encoding an ASH1L polypeptide. The method can comprise determining that the cell comprises the mutated version of nucleic acid encoding an OBSCN polypeptide. The method can comprise determining that the cell comprises the mutated version of nucleic acid encoding a TTRAP polypeptide. The method can comprise determining that the cell comprises the mutated version of nucleic acid encoding a LRP2 polypeptide. The method can comprise determining that the cell comprises the mutated version of nucleic acid encoding a TP53 polypeptide, the mutated version of nucleic acid encoding a CELSR1 polypeptide, the mutated version of nucleic acid encoding a CASP8 polypeptide, and the mutated version of nucleic acid encoding a KMT2D polypeptide.

In another aspect, this document features a method for identifying a mammal as having OLP that is likely to follow a benign course. The method comprises (or consists essentially of, or consists of) (a) determining that a cell of the OLP lacks a mutant version of nucleic acid encoding a TP53 polypeptide, a mutated version of nucleic acid encoding a CELSR1 polypeptide, a mutated version of nucleic acid encoding a CASP8 polypeptide, and a mutated version of nucleic acid encoding a KMT2D polypeptide, and (b) classifying the mammal as having the OLP. The mammal can be a human. The cell can be within a cytology brushing sample. The cell can be a squamous epithelial cell or a stromal cell affected by the OLP. The method can further comprise determining that the cell lacks a mutated version of nucleic acid encoding a TENM3 polypeptide, a mutated version of nucleic acid encoding an ASH1L polypeptide, a mutated version of nucleic acid encoding an OBSCN polypeptide, a mutated version of nucleic acid encoding a TTRAP polypeptide, and a mutated version of nucleic acid encoding a LRP2 polypeptide,

In another aspect, this document features a method for treating a mammal having OLP. The method comprises (or consists essentially of, or consists of) (a) determining that a cell of the OLP comprises a mutant version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, or a mutated version of a nucleic acid encoding a LRP2 polypeptide, thereby identifying the OLP as being likely to progress to OSCC, and (b) administering to the mammal an immunosuppressive or immunomodulatory agent. The mammal can be a human. The cell can be within a cytology brushing sample. The cell can be a squamous epithelial cell or a stromal cell affected by the OLP. The method can comprise determining that the cell comprises the mutant version of nucleic acid encoding a TP53 polypeptide. The method can comprise determining that the cell comprises the mutated version of nucleic acid encoding a CELSR1 polypeptide. The method can comprise determining that the cell comprises the mutated version of nucleic acid encoding a CASP8 polypeptide. The method can comprise determining that the cell comprises the mutated version of nucleic acid encoding a KMT2D polypeptide. The method can comprise determining that the cell comprises the mutant version of nucleic acid encoding a TP53 polypeptide, the mutated version of nucleic acid encoding a CELSR1 polypeptide, the mutated version of nucleic acid encoding a CASP8 polypeptide, and the mutated version of nucleic acid encoding a KMT2D polypeptide. The method can comprise determining that the cell comprises the mutated version of nucleic acid encoding a TENM3 polypeptide. The method can include determining that the cell comprises the mutated version of nucleic acid encoding an ASH1L polypeptide. The method can comprise determining that the cell comprises the mutated version of nucleic acid encoding an OBSCN polypeptide. The method can comprise determining that the cell comprises the mutated version of nucleic acid encoding a TTRAP polypeptide. The method can comprise determining that the cell comprises the mutated version of nucleic acid encoding a LRP2 polypeptide. The immunosuppressive or immunomodulatory agent can comprise a corticosteroid, dapsone, azathioprine, tacrolimus, mycophenolate mofetil, or a biologic agent.

In another aspect, this document features a method for treating OLP, where the method comprises (or consists essentially of, or consists of) administering, to a mammal identified as having a cell of the OLP comprising a mutant version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, or a mutated version of a nucleic acid encoding a LRP2 polypeptide, an immunosuppressive or immunomodulatory agent. The mammal can be a human. The cell can be a squamous epithelial cell or a stromal cell affected by the OLP. The mammal can be a mammal identified as having as cell of the OLP comprising the mutant version of nucleic acid encoding a TP53 polypeptide. The mammal can be a mammal identified as having as cell of the OLP comprising the mutant version of nucleic acid encoding a CELSR1 polypeptide. The mammal can be a mammal identified as having as cell of the OLP comprising the mutant version of nucleic acid encoding a CASP8 polypeptide. The mammal can be a mammal identified as having as cell of the OLP comprising the mutant version of nucleic acid encoding a KMT2D polypeptide. The mammal can be a mammal identified as having as cell of the OLP comprising the mutant version of nucleic acid encoding a TP53 polypeptide, the mutated version of nucleic acid encoding a CELSR1 polypeptide, the mutated version of nucleic acid encoding a CASP8 polypeptide, and the mutated version of nucleic acid encoding a KMT2D polypeptide. The method can comprise determining that the cell comprises the mutated version of nucleic acid encoding a TENM3 polypeptide. The method can include determining that the cell comprises the mutated version of nucleic acid encoding an ASH1L polypeptide. The method can comprise determining that the cell comprises the mutated version of nucleic acid encoding an OBSCN polypeptide. The method can comprise determining that the cell comprises the mutated version of nucleic acid encoding a TTRAP polypeptide. The method can comprise determining that the cell comprises the mutated version of nucleic acid encoding a LRP2 polypeptide. The immunosuppressive or immunomodulatory agent can comprise a corticosteroid, dapsone, azathioprine, tacrolimus, mycophenolate mofetil, or a biologic agent.

In another aspect, this document features a method for identifying a mammal as having OLP that is likely to progress to OSCC, where the method includes (or consists essentially of, or consists of) (a) determining that a cell of said OLP comprises elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB, and (b) classifying said mammal as having said OLP. The mammal can be a human. The cell can be within a cytology brushing sample. The cell can be a squamous epithelial cell or a stromal cell affected by said indolent OLP. The method can include determining that said cell comprises said elevated expression of CA1. The method can include determining that said cell comprises said elevated expression of TNNT3. The method can include determining that said cell comprises said elevated expression of SYNM. The method can include determining that said cell comprises said elevated expression of MB. The method can include determining that said cell comprises said elevated expression of MYH4. The method can include determining that said cell comprises said elevated expression of ST13P4. The method can include determining that said cell comprises said elevated expression of MYLPF. The method can include determining that said cell comprises said elevated expression of MYH1. The method can include determining that said cell comprises said elevated expression of TNNC2. The method can include determining that said cell comprises said elevated expression of SRPRB. The method can include determining that said cell comprises said elevated expression of CA1, said elevated expression of TNNT3, said elevated expression of SYNM, said elevated expression of MB, said elevated expression of MYH4, said elevated expression of ST13P4, said elevated expression of MYLPF, said elevated expression of MYH1, said elevated expression of TNNC2, and said elevated expression of SRPRB. The elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB can include elevated expression of a CA1 mRNA, elevated expression of a TNNT3 mRNA, elevated expression of a SYNM mRNA, elevated expression of a MB mRNA, elevated expression of a MYH4 mRNA, elevated expression of a ST13P4 mRNA, elevated expression of a MYLPF mRNA, elevated expression of a MYH1 mRNA, elevated expression of a TNNC2 mRNA, elevated expression of a SRPRB mRNA, or a combination thereof. The elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB can include elevated expression of a CA1 polypeptide, elevated expression of a TNNT3 polypeptide, elevated expression of a SYNM polypeptide, elevated expression of a MB polypeptide, elevated expression of a MYH4 polypeptide, elevated expression of a MYLPF polypeptide, elevated expression of a MYH1 polypeptide, elevated expression of a TNNC2 polypeptide, elevated expression of a SRPRB polypeptide, or a combination thereof.

In another aspect, this document features a method for identifying a mammal as having OLP that is likely to follow a benign course, where the method includes (or consists essentially of, or consists of) (a) determining that a cell of said OLP does not contain elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, and elevated expression of SRPRB, and (b) classifying said mammal as having said OLP. The mammal can be a human. The cell can be within a cytology brushing sample. The cell can be a squamous epithelial cell or a stromal cell affected by said OLP.

In yet another aspect, this document features a method for treating a mammal having OLP, where the method comprises (or consists essentially of, or consists of) (a) determining that a cell of said OLP comprises elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB, thereby identifying said indolent OLP as being likely to progress to transforming OLP, and (b) administering to said mammal an immunosuppressive or immunomodulatory agent. The mammal can be a human. The cell can be within a cytology brushing sample. The cell can be a squamous epithelial cell or a stromal cell affected by said indolent OLP. The method can include determining that said cell comprises said elevated expression of CA1. The method can include determining that said cell comprises said elevated expression of TNNT3. The method can include determining that said cell comprises said elevated expression of SYNM. The method can include determining that said cell comprises said elevated expression of MB. The method can include determining that said cell comprises said elevated expression of MYH4. The method can include determining that said cell comprises said elevated expression of ST13P4. The method can include determining that said cell comprises said elevated expression of MYLPF. The method can include determining that said cell comprises said elevated expression of MYH1. The method can include determining that said cell comprises said elevated expression of TNNC2. The method can include determining that said cell comprises said elevated expression of SRPRB. The method can include determining that said cell comprises said elevated expression of CA1, said elevated expression of TNNT3, said elevated expression of SYNM, said elevated expression of MB, said elevated expression of MYH4, said elevated expression of ST13P4, said elevated expression of MYLPF, said elevated expression of MYH1, said elevated expression of TNNC2, and said elevated expression of SRPRB. The elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB can include elevated expression of a CA1 mRNA, elevated expression of a TNNT3 mRNA, elevated expression of a SYNM mRNA, elevated expression of a MB mRNA, elevated expression of a MYH4 mRNA, elevated expression of a ST13P4 mRNA, elevated expression of a MYLPF mRNA, elevated expression of a MYH1 mRNA, elevated expression of a TNNC2 mRNA, elevated expression of a SRPRB mRNA, or a combination thereof. The elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB can include elevated expression of a CA1 polypeptide, elevated expression of a TNNT3 polypeptide, elevated expression of a SYNM polypeptide, elevated expression of a MB polypeptide, elevated expression of a MYH4 polypeptide, elevated expression of a MYLPF polypeptide, elevated expression of a MYH1 polypeptide, elevated expression of a TNNC2 polypeptide, elevated expression of a SRPRB polypeptide, or a combination thereof. The immunosuppressive or immunomodulatory agent can include a corticosteroid, dapsone, azathioprine, tacrolimus, mycophenolate mofetil, or a biologic agent.

This document also features a method for treating OLP, where the method comprises (or consists essentially of, or consists if) administering, to a mammal identified as having a cell of said OLP comprising elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB, an immunosuppressive or immunomodulatory agent. The mammal can be a human. The cell can be a squamous epithelial cell or a stromal cell affected by said OLP. The mammal can be a mammal identified as having as cell of said OLP comprising said elevated expression of CA1. The mammal can be a mammal identified as having as cell of said OLP comprising said elevated expression of TNNT3. The mammal can be a mammal identified as having as cell of said OLP comprising said elevated expression of SYNM. The mammal can be a mammal identified as having as cell of said OLP comprising said elevated expression of MB. The mammal can be a mammal identified as having as cell of said OLP comprising said elevated expression of MYH4. The mammal can be a mammal identified as having as cell of said OLP comprising said elevated expression of ST13P4. The mammal can be a mammal identified as having as cell of said OLP comprising said elevated expression of MYLPF. The mammal can be a mammal identified as having as cell of said OLP comprising said elevated expression of MYH1. The mammal can be a mammal identified as having as cell of said OLP comprising said elevated expression of TNNC2. The mammal can be a mammal identified as having as cell of said OLP comprising said elevated expression of SRPRB. The mammal can be a mammal identified as having as cell of said OLP comprising said elevated expression of CA1, said elevated expression of TNNT3, said elevated expression of SYNM, said elevated expression of MB, said elevated expression of MYH4, said elevated expression of ST13P4, said elevated expression ofMYLPF, said elevated expression of MYH1, said elevated expression of TNNC2, and said elevated expression of SRPRB. The elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB can include elevated expression of a CA1 mRNA, elevated expression of a TNNT3 mRNA, elevated expression of a SYNM mRNA, elevated expression of a MB mRNA, elevated expression of a MYH4 mRNA, elevated expression of a ST13P4 mRNA, elevated expression of a MYLPF mRNA, elevated expression of a MYH1 mRNA, elevated expression of a TNNC2 mRNA, elevated expression of a SRPRB mRNA, or a combination thereof. The elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB can include elevated expression of a CA1 polypeptide, elevated expression of a TNNT3 polypeptide, elevated expression of a SYNM polypeptide, elevated expression of a MB polypeptide, elevated expression of a MYH4 polypeptide, elevated expression of a MYLPF polypeptide, elevated expression of a MYH1 polypeptide, elevated expression of a TNNC2 polypeptide, elevated expression of a SRPRB polypeptide, or a combination thereof. The immunosuppressive or immunomodulatory agent can include a corticosteroid, dapsone, azathioprine, tacrolimus, mycophenolate mofetil, or a biologic agent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description, and from the claims.

### DESCRIPTION OF THE DRAWINGS

FIGS. 1A-IC are representative histopathology images showing indolent oral lichen planus (OLP) with band-like lymphocytic infiltrate, basal liquefactive degeneration and lymphocyte exocytosis (hematoxylin-and-eosin stain; original magnification 10×) (FIG. 1A); transforming OLP showing similar characteristics but from area of OLP adjacent to oral squamous cell carcinoma (OSCC) (10×) (FIG. 1B); and OSCC with marked cytological atypia and infiltrative stromal invasion (4×) (FIG. 1C).

FIG. 2 shows variants seen in transforming oral lichen planus (OLP) and oral squamous cell carcinoma (OSCC) but not in indolent OLP, in at least 4 samples or 20% frequency. Affected genes are listed in rows, while individual samples are represented in columns. Mutation types are indicated below the graph.

FIG. 3 is a schematic summarizing key somatic mutations in oral biopsy samples of indolent oral lichen planus (OLP), transforming OLP, or oral squamous cell carcinoma (OSCC), with frequency>20%.

FIG. 4 is a schematic showing a potential pathway for carcinogenesis in transforming OLP and OSCC for the four most frequently mutated genes. Specifically, TP53 mutation allows evasion of the kill response after DNA damage; CASP8 allows further evasion of apoptosis even if another gatekeeper, e.g., p63 or p21 invokes a kill response to DNA damage signaling cell senescence; and KMT2D prevents the activation of the DNA damage repair response all leading to further accumulation of DNA damage. This further damage may confer further proliferative and carcinogenic activity, including loss of CELSR1 which interrupts cell adhesion to allow invasion.

FIGS. 5A-5C are representative photomicrographs of transforming OLP, taken from areas marginal to but not immediately adjacent to oral squamous cell carcinoma, with FIG. 5A showing low power sections, FIG. 5B showing a close up of squamous cell carcinoma, and FIG. 5C showing OLP.

FIG. 6A is a volcano plot showing proteins overexpressed (to the right of the X-axis zero point) and underexpressed (to the left of the X-axis zero point) in transforming OLP compared to indolent OLP. The plot identifies proteins that have a 1-fold change (FC) and false discovery rate (FDR)≤0.5. FIG. 6B is a volcano plot showing proteins overexpressed (to the right of the X-axis zero point) and underexpressed (to the left of the X-axis zero point) in indolent OLP and transforming OLP compared to normal oral mucosa (NOM). The plot identifies proteins that have a 1-FC and FDR 0.5.

FIG. 7A is a graph showing the most significant canonical pathways for differentially expressed proteins in transforming OLP compared to indolent OLP that have a FC≥1 or ≤−1 and FDR≤0.5. FIG. 7B is a graph showing the most significant canonical pathways for differentially expressed proteins in indolent OLP and transforming OLP compared to normal oral mucosa that have a FC≥1 or ≤−1 and FDR≤0.5.

### DETAILED DESCRIPTION

This document provides methods and materials for identifying and/or treating a mammal (e.g., a human) having OLP that is likely to proceed to OSCC. This document also provides methods and materials for identifying and/or treating a mammal (e.g., a human) having OLP that is likely to follow a benign course of the disorder.

Any appropriate mammal having OLP can be identified as having an OLP that is likely to proceed to OSCC or that is likely to follow a benign course of the disorder. For example, humans and other primates such as monkeys having OLP can be identified as having an OLP that is likely to proceed to OSCC or that is likely to follow a benign course of the disorder. In some cases, dogs, cats, horses, cows, pigs, sheep, mice, or rats having OLP can be identified as having an OLP that is likely to proceed to OSCC or that is likely to follow a benign course of the disorder as described herein.

As described herein, a mammal (e.g., a human) having OLP can be identified as having an OLP that is likely to proceed to OSCC by determining that cells of the OLP have a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or a combination thereof. In some cases, a mammal (e.g., a human) having OLP can be identified as having an OLP that is likely to follow a benign course of the disorder by determining that cells of the OLP lack a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and a mutated version of a nucleic acid encoding a LRP2 polypeptide.

Any appropriate OLP sample can be obtained and assessed to determine if the OLP contains cells having or lacking a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and/or a mutated version of a nucleic acid encoding a LRP2 polypeptide. For example, a tissue biopsy of OLP can be obtained and assessed. In some cases, OLP lesional cells can be obtained via a biopsy, brushing, swabbing, scraping, or a saliva collection.

Once cells of OLP are obtained from a mammal (e.g., a human), the cells can be assessed to determine if they have or lack a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and/or a mutated version of a nucleic acid encoding a LRP2 polypeptide. Any appropriate method can be used to determine the presence or absence of a mutation in nucleic acid encoding a TP53 polypeptide, nucleic acid encoding a CELSR1 polypeptide, nucleic acid encoding a CASP8 polypeptide, nucleic acid encoding a KMT2D polypeptide, nucleic acid encoding a TENM3 polypeptide, nucleic acid encoding an ASH1L polypeptide, nucleic acid encoding an OBSCN polypeptide, nucleic acid encoding a TTRAP polypeptide, and/or nucleic acid encoding a LRP2 polypeptide. For example, nucleic acid sequencing techniques (e.g., next generation sequencing), nucleic acid-based mutation detection assays (e.g., TaqMan mutation detection assays), and enzymatic-based mutation detection assays can be used to detect the presence or absence of a mutation in nucleic acid encoding a TP53 polypeptide, nucleic acid encoding a CELSR1 polypeptide, nucleic acid encoding a CASP8 polypeptide, nucleic acid encoding a KMT2D polypeptide, nucleic acid encoding a TENM3 polypeptide, nucleic acid encoding an ASH1L polypeptide, nucleic acid encoding an OBSCN polypeptide, nucleic acid encoding a TTRAP polypeptide, and/or nucleic acid encoding a LRP2 polypeptide. In some cases, TP53, CELSR1, CASP8, KMT2D, TENM3, ASH1L, OBSCN, TTRAP, and/or LRP2 polypeptides can be assessed to determine the cells have or lack a mutated version of nucleic acid encoding one or more of those polypeptides. For example, mass spectrometry or Edman degradation can be used to identify the presence or absence of a mutation in a TP53, CELSR1, CASP8, KMT2D, TENM3, ASH1L, OBSCN, TTRAP, and/or LRP2 polypeptide.

The presence or absence of a mutation in nucleic acid encoding a TP53 polypeptide, nucleic acid encoding a CELSR1 polypeptide, nucleic acid encoding a CASP8 polypeptide, nucleic acid encoding a KMT2D polypeptide, nucleic acid encoding a TENM3 polypeptide, nucleic acid encoding an ASH1L polypeptide, nucleic acid encoding an OBSCN polypeptide, nucleic acid encoding a TTRAP polypeptide, and/or nucleic acid encoding a LRP2 polypeptide is determined as compared to the wild-type versions of these nucleic acids from the appropriate species. For example, when assessing a sample from a human for cells having a mutated version of human nucleic acid encoding a TP53 polypeptide, then the wild-type human nucleic acid encoding that TP53 polypeptide is used as the reference to determine the presence or absence of a mutation. When assessing a sample from a human for cells having a mutated version of human nucleic acid encoding a TP53 polypeptide, the wild-type human nucleic acid encoding a TP53 polypeptide that can be used as a reference can have the nucleic acid sequence set forth in NCBI Reference Sequence NG_017013 (accession number version NG_017013.2). In some cases, a mutated version of a nucleic acid encoding a TP53 polypeptide (e.g., a human nucleic acid encoding a TP53 polypeptide) can be a nucleic acid having one or more mutations in the coding sequence. In some cases, a mutated version of a nucleic acid encoding a TP53 polypeptide (e.g., a human nucleic acid encoding a TP53 polypeptide) can be a nucleic acid having one or more mutations that result in one or more amino acid changes in the TP53 polypeptide. For example, when assessing a sample from a human for cells having a mutated version of human nucleic acid encoding a TP53 polypeptide with one or more mutations in the coding sequence, the wild-type human nucleic acid encoding a TP53 polypeptide that can be used as a reference can have the TP53 polypeptide-encoding nucleic acid sequence set forth in NCBI Reference Sequence NG_017013 (accession number version NG_017013.2).

When assessing a sample from a human for cells having a mutated version of human nucleic acid encoding a CELSR1 polypeptide, the wild-type human nucleic acid encoding a CELSR1 polypeptide that can be used as a reference can have the nucleic acid sequence set forth in NCBI Reference Sequence NG_030466 (accession number version NG_030466.2). In some cases, a mutated version of a nucleic acid encoding a CELSR1 polypeptide (e.g., a human nucleic acid encoding a CELSR1 polypeptide) can be a nucleic acid having one or more mutations in the coding sequence. In some cases, a mutated version of a nucleic acid encoding a CELSR1 polypeptide (e.g., a human nucleic acid encoding a CELSR1 polypeptide) can be a nucleic acid having one or more mutations that result in one or more amino acid changes in the CELSR1 polypeptide. For example, when assessing a sample from a human for cells having a mutated version of human nucleic acid encoding a CELSR1 polypeptide with one or more mutations in the coding sequence, the wild-type human nucleic acid encoding a CELSR1 polypeptide that can be used as a reference can have the CELSR1 polypeptide-encoding nucleic acid sequence set forth in NCBI Reference Sequence NG_030466 (accession number version NG_030466.2).

When assessing a sample from a human for cells having a mutated version of human nucleic acid encoding a CASP8 polypeptide, the wild-type human nucleic acid encoding a CASP8 polypeptide that can be used as a reference can have the nucleic acid sequence set forth in NCBI Reference Sequence NG_007497 (accession number version NG_007497.1). In some cases, a mutated version of a nucleic acid encoding a CASP8 polypeptide (e.g., a human nucleic acid encoding a CASP8 polypeptide) can be a nucleic acid having one or more mutations in the coding sequence. In some cases, a mutated version of a nucleic acid encoding a CASP8 polypeptide (e.g., a human nucleic acid encoding a CASP8 polypeptide) can be a nucleic acid having one or more mutations that result in one or more amino acid changes in the CASP8 polypeptide. For example, when assessing a sample from a human for cells having a mutated version of human nucleic acid encoding a CASP8 polypeptide with one or more mutations in the coding sequence, the wild-type human nucleic acid encoding a CASP8 polypeptide that can be used as a reference can have the CASP8 polypeptide-encoding nucleic acid sequence set forth in NCBI Reference Sequence NG_007497 (accession number version NG_007497.1).

When assessing a sample from a human for cells having a mutated version of human nucleic acid encoding a KMT2D polypeptide, the wild-type human nucleic acid encoding a KMT2D polypeptide that can be used as a reference can have the nucleic acid sequence set forth in NCBI Reference Sequence NG_027827 (accession number version NG_027827.1). In some cases, a mutated version of a nucleic acid encoding a KMT2D polypeptide (e.g., a human nucleic acid encoding a KMT2D polypeptide) can be a nucleic acid having one or more mutations in the coding sequence. In some cases, a mutated version of a nucleic acid encoding a KMT2D polypeptide (e.g., a human nucleic acid encoding a KMT2D polypeptide) can be a nucleic acid having one or more mutations that result in one or more amino acid changes in the KMT2D polypeptide. For example, when assessing a sample from a human for cells having a mutated version of human nucleic acid encoding a KMT2D polypeptide with one or more mutations in the coding sequence, the wild-type human nucleic acid encoding a KMT2D polypeptide that can be used as a reference can have the KMT2D polypeptide-encoding nucleic acid sequence set forth in NCBI Reference Sequence NG_027827 (accession number version NG_027827.1).

When assessing a sample from a human for cells having a mutated version of human nucleic acid encoding a TENM3 polypeptide, the wild-type human nucleic acid encoding a TENM3 polypeptide that can be used as a reference can have the nucleic acid sequence set forth in NCBI Reference Sequence NG_042859 (accession number version NG_042859.1). In some cases, a mutated version of a nucleic acid encoding a TENM3 polypeptide (e.g., a human nucleic acid encoding a TENM3 polypeptide) can be a nucleic acid having one or more mutations in the coding sequence. In some cases, a mutated version of a nucleic acid encoding a TENM3 polypeptide (e.g., a human nucleic acid encoding a TENM3 polypeptide) can be a nucleic acid having one or more mutations that result in one or more amino acid changes in the TENM3 polypeptide. For example, when assessing a sample from a human for cells having a mutated version of human nucleic acid encoding a TENM3 polypeptide with one or more mutations in the coding sequence, the wild-type human nucleic acid encoding a TENM3 polypeptide that can be used as a reference can have the TENM3 polypeptide-encoding nucleic acid sequence set forth in NCBI Reference Sequence NG_042859 (accession number version NG_042859.1).

When assessing a sample from a human for cells having a mutated version of human nucleic acid encoding a ASH1L polypeptide, the wild-type human nucleic acid encoding a ASH1L polypeptide that can be used as a reference can have the nucleic acid sequence set forth in NCBI Reference Sequence NC_000001 (accession number version NC_000001.11). In some cases, a mutated version of a nucleic acid encoding a ASH1L polypeptide (e.g., a human nucleic acid encoding a ASH1L polypeptide) can be a nucleic acid having one or more mutations in the coding sequence. In some cases, a mutated version of a nucleic acid encoding a ASH1L polypeptide (e.g., a human nucleic acid encoding a ASH1L polypeptide) can be a nucleic acid having one or more mutations that result in one or more amino acid changes in the ASH1L polypeptide. For example, when assessing a sample from a human for cells having a mutated version of human nucleic acid encoding a ASH1L polypeptide with one or more mutations in the coding sequence, the wild-type human nucleic acid encoding a ASH1L polypeptide that can be used as a reference can have the ASH1L polypeptide-encoding nucleic acid sequence set forth in NCBI Reference Sequence NC_000001 (accession number version NC_000001.11).

When assessing a sample from a human for cells having a mutated version of human nucleic acid encoding a OBSCN polypeptide, the wild-type human nucleic acid encoding a OBSCN polypeptide that can be used as a reference can have the nucleic acid sequence set forth in NCBI Reference Sequence NG_032122 (accession number version NG_032122.1). In some cases, a mutated version of a nucleic acid encoding a OBSCN polypeptide (e.g., a human nucleic acid encoding a OBSCN polypeptide) can be a nucleic acid having one or more mutations in the coding sequence. In some cases, a mutated version of a nucleic acid encoding a OBSCN polypeptide (e.g., a human nucleic acid encoding a OBSCN polypeptide) can be a nucleic acid having one or more mutations that result in one or more amino acid changes in the OBSCN polypeptide. For example, when assessing a sample from a human for cells having a mutated version of human nucleic acid encoding a OBSCN polypeptide with one or more mutations in the coding sequence, the wild-type human nucleic acid encoding a OBSCN polypeptide that can be used as a reference can have the OBSCN polypeptide-encoding nucleic acid sequence set forth in NCBI Reference Sequence NG_032122 (accession number version NG_032122.1).

When assessing a sample from a human for cells having a mutated version of human nucleic acid encoding a TTRAP polypeptide, the wild-type human nucleic acid encoding a TTRAP polypeptide that can be used as a reference can have the nucleic acid sequence set forth in NCBI Reference Sequence NG_052787 (accession number version NG_052787.1). In some cases, a mutated version of a nucleic acid encoding a TTRAP polypeptide (e.g., a human nucleic acid encoding a TTRAP polypeptide) can be a nucleic acid having one or more mutations in the coding sequence. In some cases, a mutated version of a nucleic acid encoding a TTRAP polypeptide (e.g., a human nucleic acid encoding a TTRAP polypeptide) can be a nucleic acid having one or more mutations that result in one or more amino acid changes in the TTRAP polypeptide. For example, when assessing a sample from a human for cells having a mutated version of human nucleic acid encoding a TTRAP polypeptide with one or more mutations in the coding sequence, the wild-type human nucleic acid encoding a TTRAP polypeptide that can be used as a reference can have the TTRAP polypeptide-encoding nucleic acid sequence set forth in NCBI Reference Sequence NG_052787 (accession number version NG_052787.1).

When assessing a sample from a human for cells having a mutated version of human nucleic acid encoding a LRP2 polypeptide, the wild-type human nucleic acid encoding a LRP2 polypeptide that can be used as a reference can have the nucleic acid sequence set forth in NCBI Reference Sequence NG_012634 (accession number version NG_012634.1). In some cases, a mutated version of a nucleic acid encoding a LRP2 polypeptide (e.g., a human nucleic acid encoding a LRP2 polypeptide) can be a nucleic acid having one or more mutations in the coding sequence. In some cases, a mutated version of a nucleic acid encoding a LRP2 polypeptide (e.g., a human nucleic acid encoding a LRP2 polypeptide) can be a nucleic acid having one or more mutations that result in one or more amino acid changes in the LRP2 polypeptide. For example, when assessing a sample from a human for cells having a mutated version of human nucleic acid encoding a LRP2 polypeptide with one or more mutations in the coding sequence, the wild-type human nucleic acid encoding a LRP2 polypeptide that can be used as a reference can have the LRP2 polypeptide-encoding nucleic acid sequence set forth in NCBI Reference Sequence NG_012634 (accession number version NG_012634.1). TABLE 3 lists examples of mutations that can be detected and used to identify OLP as being likely to proceed to OSCC.

As described herein, a mammal (e.g., a human) having OLP can be identified as having an OLP that is likely to proceed to OSCC by determining that cells of the OLP have a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or a combination thereof. For example, a mammal (e.g., a human) having OLP can be identified as having an OLP that is likely to proceed to OSCC by determining that cells of the OLP have a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, or a mutated version of a nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a CELSR1 polypeptide. In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a CASP8 polypeptide. In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a KMT2D polypeptide. In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a TENM3 polypeptide. In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a ASH1L polypeptide. In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a OBSCN polypeptide. In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a TTRAP polypeptide. In some cases, a mammal (e.g., a human) having OLP can be identified as having an OLP that is likely to proceed to OSCC by determining that cells of the OLP have a mutated version of nucleic acid encoding a TP53 polypeptide and a mutated version of nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a CELSR1 polypeptide and a mutated version of nucleic acid encoding a CASP8 polypeptide. In some cases, a mammal (e.g., a human) having OLP can be identified as having an OLP that is likely to proceed to OSCC by determining that cells of the OLP have a mutated version of nucleic acid encoding a CELSR1 polypeptide and a mutated version of nucleic acid encoding a KMT2D polypeptide. In some cases, a mammal (e.g., a human) having OLP can be identified as having an OLP that is likely to proceed to OSCC by determining that cells of the OLP have a mutated version of nucleic acid encoding a CELSR1 polypeptide and a mutated version of nucleic acid encoding a TENM3 polypeptide. In some cases, a mammal (e.g., a human) having OLP can be identified as having an OLP that is likely to proceed to OSCC by determining that cells of the OLP have a mutated version of nucleic acid encoding a CELSR1 polypeptide and a mutated version of nucleic acid encoding a ASH1L polypeptide. In some cases, a mammal (e.g., a human) having OLP can be identified as having an OLP that is likely to proceed to OSCC by determining that cells of the OLP have a mutated version of nucleic acid encoding a CELSR1 polypeptide and a mutated version of nucleic acid encoding a OBSCN polypeptide. In some cases, a mammal (e.g., a human) having OLP can be identified as having an OLP that is likely to proceed to OSCC by determining that cells of the OLP have a mutated version of nucleic acid encoding a CELSR1 polypeptide and a mutated version of nucleic acid encoding a TTRAP polypeptide. In some cases, a mammal (e.g., a human) having OLP can be identified as having an OLP that is likely to proceed to OSCC by determining that cells of the OLP have a mutated version of nucleic acid encoding a CELSR1 polypeptide and a mutated version of nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a CASP8 polypeptide and a mutated version of nucleic acid encoding a KMT2D polypeptide. In some cases, a mammal (e.g., a human) having OLP can be identified as having an OLP that is likely to proceed to OSCC by determining that cells of the OLP have a mutated version of nucleic acid encoding a CASP8 polypeptide and a mutated version of nucleic acid encoding a TENM3 polypeptide. In some cases, a mammal (e.g., a human) having OLP can be identified as having an OLP that is likely to proceed to OSCC by determining that cells of the OLP have a mutated version of nucleic acid encoding a CASP8 polypeptide and a mutated version of nucleic acid encoding a ASH1L polypeptide. In some cases, a mammal (e.g., a human) having OLP can be identified as having an OLP that is likely to proceed to OSCC by determining that cells of the OLP have a mutated version of nucleic acid encoding a CASP8 polypeptide and a mutated version of nucleic acid encoding a OBSCN polypeptide. In some cases, a mammal (e.g., a human) having OLP can be identified as having an OLP that is likely to proceed to OSCC by determining that cells of the OLP have a mutated version of nucleic acid encoding a CASP8 polypeptide and a mutated version of nucleic acid encoding a TTRAP polypeptide. In some cases, a mammal (e.g., a human) having OLP can be identified as having an OLP that is likely to proceed to OSCC by determining that cells of the OLP have a mutated version of nucleic acid encoding a CASP8 polypeptide and a mutated version of nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a KMT2D polypeptide and a mutated version of nucleic acid encoding a TENM3 polypeptide. In some cases, a mammal (e.g., a human) having OLP can be identified as having an OLP that is likely to proceed to OSCC by determining that cells of the OLP have a mutated version of nucleic acid encoding a KMT2D polypeptide and a mutated version of nucleic acid encoding a ASH1L polypeptide. In some cases, a mammal (e.g., a human) having OLP can be identified as having an OLP that is likely to proceed to OSCC by determining that cells of the OLP have a mutated version of nucleic acid encoding a KMT2D polypeptide and a mutated version of nucleic acid encoding a OBSCN polypeptide. In some cases, a mammal (e.g., a human) having OLP can be identified as having an OLP that is likely to proceed to OSCC by determining that cells of the OLP have a mutated version of nucleic acid encoding a KMT2D polypeptide and a mutated version of nucleic acid encoding a TTRAP polypeptide. In some cases, a mammal (e.g., a human) having OLP can be identified as having an OLP that is likely to proceed to OSCC by determining that cells of the OLP have a mutated version of nucleic acid encoding a KMT2D polypeptide and a mutated version of nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a TENM3 polypeptide and a mutated version of nucleic acid encoding a ASH1L polypeptide. In some cases, a mammal (e.g., a human) having OLP can be identified as having an OLP that is likely to proceed to OSCC by determining that cells of the OLP have a mutated version of nucleic acid encoding a TENM3 polypeptide and a mutated version of nucleic acid encoding a OBSCN polypeptide. In some cases, a mammal (e.g., a human) having OLP can be identified as having an OLP that is likely to proceed to OSCC by determining that cells of the OLP have a mutated version of nucleic acid encoding a TENM3 polypeptide and a mutated version of nucleic acid encoding a TTRAP polypeptide. In some cases, a mammal (e.g., a human) having OLP can be identified as having an OLP that is likely to proceed to OSCC by determining that cells of the OLP have a mutated version of nucleic acid encoding a TENM3 polypeptide and a mutated version of nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a ASH1L polypeptide and a mutated version of nucleic acid encoding a OBSCN polypeptide. In some cases, a mammal (e.g., a human) having OLP can be identified as having an OLP that is likely to proceed to OSCC by determining that cells of the OLP have a mutated version of nucleic acid encoding a ASH1L polypeptide and a mutated version of nucleic acid encoding a TTRAP polypeptide. In some cases, a mammal (e.g., a human) having OLP can be identified as having an OLP that is likely to proceed to OSCC by determining that cells of the OLP have a mutated version of nucleic acid encoding a ASH1L polypeptide and a mutated version of nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a OBSCN polypeptide and a mutated version of nucleic acid encoding a TTRAP polypeptide. In some cases, a mammal (e.g., a human) having OLP can be identified as having an OLP that is likely to proceed to OSCC by determining that cells of the OLP have a mutated version of nucleic acid encoding a OBSCN polypeptide and a mutated version of nucleic acid encoding a LRP2 polypeptide. In some cases, a mammal (e.g., a human) having OLP can be identified as having an OLP that is likely to proceed to OSCC by determining that cells of the OLP have a mutated version of nucleic acid encoding a TTRAP polypeptide and a mutated version of nucleic acid encoding a LRP2 polypeptide.

In some cases, a mammal (e.g., a human) having OLP can be identified as having an OLP that is likely to proceed to OSCC by determining that cells of the OLP have a mutated version of nucleic acid encoding a TP53 polypeptide, a mutated version of nucleic acid encoding a CELSR1 polypeptide, and a mutated version of nucleic acid encoding a CASP8 polypeptide. In some cases, a mammal (e.g., a human) having OLP can be identified as having an OLP that is likely to proceed to OSCC by determining that cells of the OLP have a mutated version of nucleic acid encoding a TP53 polypeptide, a mutated version of nucleic acid encoding a CELSR1 polypeptide, and a mutated version of nucleic acid encoding a KMT2D polypeptide. In some cases, a mammal (e.g., a human) having OLP can be identified as having an OLP that is likely to proceed to OSCC by determining that cells of the OLP have a mutated version of nucleic acid encoding a CELSR1 polypeptide, a mutated version of nucleic acid encoding a CASP8 polypeptide, and a mutated version of nucleic acid encoding a KMT2D polypeptide. In some cases, a mammal (e.g., a human) having OLP can be identified as having an OLP that is likely to proceed to OSCC by determining that cells of the OLP have a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and a mutated version of a nucleic acid encoding a LRP2 polypeptide.

A mutated version of nucleic acid encoding a TP53 polypeptide can have one, two, three, four, five, or more mutations as compared to the wild-type version of nucleic acid encoding a TP53 polypeptide for the species being assessed. A mutated version of nucleic acid encoding a CELSR1 polypeptide can have one, two, three, four, five, or more mutations as compared to the wild-type version of nucleic acid encoding a CELSR1 polypeptide for the species being assessed. A mutated version of nucleic acid encoding a CASP8 polypeptide can have one, two, three, four, five, or more mutations as compared to the wild-type version of nucleic acid encoding a CASP8 polypeptide for the species being assessed. A mutated version of nucleic acid encoding a KMT2D polypeptide can have one, two, three, four, five, or more mutations as compared to the wild-type version of nucleic acid encoding a KMT2D polypeptide for the species being assessed. A mutated version of nucleic acid encoding a TENM3 polypeptide can have one, two, three, four, five, or more mutations as compared to the wild-type version of nucleic acid encoding a TENM3 polypeptide for the species being assessed. A mutated version of nucleic acid encoding a ASH1L polypeptide can have one, two, three, four, five, or more mutations as compared to the wild-type version of nucleic acid encoding a ASH1L polypeptide for the species being assessed. A mutated version of nucleic acid encoding a OBSCN polypeptide can have one, two, three, four, five, or more mutations as compared to the wild-type version of nucleic acid encoding a OBSCN polypeptide for the species being assessed. A mutated version of nucleic acid encoding a TTRAP polypeptide can have one, two, three, four, five, or more mutations as compared to the wild-type version of nucleic acid encoding a TTRAP polypeptide for the species being assessed. A mutated version of nucleic acid encoding a LRP2 polypeptide can have one, two, three, four, five, or more mutations as compared to the wild-type version of nucleic acid encoding a LRP2 polypeptide for the species being assessed.

Once a mammal (e.g., a human) is identified as having cells containing a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or a combination thereof, the mammal can be classified as having OLP that is likely to proceed to OSCC. For example, a human identified as having cells (e.g., oral squamous epithelial or stromal cells affected by OLP) having a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, a mutated version of a nucleic acid encoding a LRP2 polypeptide, or a combination thereof can be classified as having OLP that is likely to proceed to OSCC.

In some cases, once a mammal (e.g., a human) is identified as having cells that lack a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and a mutated version of a nucleic acid encoding a LRP2 polypeptide, the mammal can be classified as having OLP that is likely to follow a benign course of the disorder. In some cases, once a mammal (e.g., a human) is identified as having cells that lack a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and a mutated version of a nucleic acid encoding a LRP2 polypeptide, the mammal can be classified as having OLP that is likely to follow a benign course of the disorder. For example, a human identified as having cells (e.g., oral squamous epithelial or stromal cells affected by OLP) lacking a mutated version of nucleic acid encoding a TP53 polypeptide, a mutated version of nucleic acid encoding a CELSR1 polypeptide, a mutated version of nucleic acid encoding a CASP8 polypeptide, and a mutated version of nucleic acid encoding a KMT2D polypeptide can be classified as having OLP that is likely to follow a benign course of the disorder. In some cases, a human identified as having cells (e.g., oral squamous epithelial or stromal cells affected by OLP) lacking a mutated version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of a nucleic acid encoding a TENM3 polypeptide, a mutated version of a nucleic acid encoding an ASH1L polypeptide, a mutated version of a nucleic acid encoding an OBSCN polypeptide, a mutated version of a nucleic acid encoding a TTRAP polypeptide, and a mutated version of a nucleic acid encoding a LRP2 polypeptide can be classified as having OLP that is likely to follow a benign course of the disorder.

As described herein, a mammal (e.g., a human) having OLP can be identified as having an OLP that is likely to proceed to OSCC by determining that cells of the OLP have elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, elevated expression of SRPRB, or a combination thereof. In some cases, a mammal (e.g., a human) having OLP can be identified as having an OLP that is likely to follow a benign course of the disorder by determining that cells of the OLP do not have elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, and elevated expression of SRPRB.

Any appropriate OLP sample can be obtained and assessed to determine if the OLP contains cells having or lacking elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, and/or elevated expression of SRPRB. For example, a tissue biopsy of OLP can be obtained and assessed. In some cases, OLP lesional cells can be obtained via a biopsy, brushing, swabbing, scraping, or a saliva collection.

Once cells of OLP are obtained from a mammal (e.g., a human), the cells can be assessed to determine if they have or lack an elevated level of CA1 expression, an elevated level of TNNT3 expression, an elevated level of SYNM expression, an elevated level of MB expression, an elevated level of MYH4 expression, an elevated level of ST13P4 expression, an elevated level of MYLPF expression, an elevated level of MYH1 expression, an elevated level of TNNC2 expression, and/or an elevated level of SRPRB expression. Any appropriate method can be used to determine the level of CA1 expression, TNNT3 expression, SYNM expression, MB expression, MYH4 expression, ST13P4 expression, MYLPF expression, expression MYH1 expression, TNNC2 expression, and/or SRPRB expression, at the mRNA level or the protein level. For example, polypeptide assessment techniques such as immunohistochemistry and enzyme-linked immunosorbent assays (ELISA) can be used to determine the level of a CA1 polypeptide, a TNNT3 polypeptide, a SYNM polypeptide, a MB polypeptide, a MYH4 polypeptide, a MYLPF polypeptide, a MYH1 polypeptide, a TNNC2 polypeptide, and/or a SRPRB polypeptide. Antibodies against CA1, TNNT3, SYNM, MB, MYH4, MYLPF, MYH1, TNNC2, and SRPRB are available from companies such as, for example, Invitrogen/ThermoFisher Scientific, BioLegend, Cell Signaling, R&D Systems, Novus Biologicals, BD Horizon, LS Bio, abcam, Proteintech, and Santa Cruz Biotech. In some cases, CA1, TNNT3, SYNM, MB, MYH4, ST13P4, MYLPF, MYH1, TNNC2, and/or SRPRB mRNAs can be assessed to determine whether the cells have or lack elevated expression of one or more of CA1, TNNT3, SYNM, MB, MYH4, ST13P4, MYLPF, MYH1, TNNC2, and/or SRPRB. For example, ST13P4 is a non-coding pseudogene, so its expression can be detected by mRNA-based methods. Non-limiting examples of methods that can be used to identify the presence or absence of elevated expression of a CA1, TNNT3, SYNM, MB, MYH4, ST13P4, MYLPF, MYH1, TNNC2, and/or SRPRB mRNA include rtPCR and Northern blotting.

A reference human CA1 polypeptide-encoding nucleic acid sequence is set forth in NCBI Reference Sequence NM_001128829 (accession number version NM_001128829.4). A reference human TNNT3 polypeptide-encoding nucleic acid sequence is set forth in NCBI Reference Sequence NM_006757 (accession number version NM_006757.4). A reference human SYNM polypeptide-encoding nucleic acid sequence is set forth in NCBI Reference Sequence NM_145728 (accession number version NM_145728.3). A reference human MB polypeptide-encoding nucleic acid sequence is set forth in NCBI Reference Sequence CR456516 (accession number version CR456516.1). A reference human MYH4 polypeptide-encoding nucleic acid sequence is set forth in NCBI Reference Sequence NM_017533 (accession number version NM_017533.2). A reference human ST13P4 nucleic acid sequence is set forth in NCBI Reference Sequence NR_002183.1 (accession number version NR_002183.1). A reference human MYLPF polypeptide-encoding nucleic acid sequence is set forth in NCBI Reference Sequence NM_013292 (accession number version NM_013292.5). A reference human MYH1 polypeptide-encoding nucleic acid sequence is set forth in NCBI Reference Sequence NM_005963 (accession number version NM_005963.4). A reference human TNNC2 polypeptide-encoding nucleic acid sequence is set forth in NCBI Reference Sequence NM_003279 (accession number version NM_003279.3). A reference human SRPRB polypeptide-encoding nucleic acid sequence is set forth in NCBI Reference Sequence NM_021203 (accession number version NM_021203.4).

The presence or absence of an elevated level of CA1, TNNT3, SYNM, MB, MYH4, ST13P4, MYLPF, MYH1, TNNC2, and/or SRPRB expression in a cell from a mammal (e.g., a human) having OLP is determined as compared to the level in a corresponding healthy cell (e.g., a healthy oral squamous epithelial or stromal cell from the human having OLP, or an oral squamous epithelial or stromal cell from a human not having OLP). For example, when assessing a sample from a human for cells having elevated expression of a CA1 polypeptide, then a corresponding sample of healthy (non-OLP) cells is used as the reference to determine the presence or absence of elevated expression of the CA1 polypeptide.

An elevated level of expression for a particular marker can be an elevated level of polypeptide or an elevated level of mRNA. An “elevated” level of a CA1 polypeptide or mRNA, a TNNT3 polypeptide or mRNA, a SYNM polypeptide or mRNA, a MB polypeptide or mRNA, a MYH4 polypeptide or mRNA, a ST13P4 mRNA, a MYLPF polypeptide or mRNA, a MYH1 polypeptide or mRNA, a TNNC2 polypeptide or mRNA, or a SRPRB polypeptide or mRNA in a cell is any level that is at least 20% (e.g., at least 30%, at least 50%, at least 100%, at least 200%, at least 300%, or at least 500%) greater than the level of that polypeptide or mRNA in a corresponding healthy cell. For example, in some cases, an elevated level of a CA1 mRNA or polypeptide in a cell from a mammal having OLP can be a level that is 900% (10-fold) greater than the level of the CA1 mRNA or polypeptide in a corresponding healthy cell. In some cases, an elevated level of CA1 in a cell from a mammal having OLP can be a level that is 1900% (20-fold) greater than the level of CA1 in a corresponding healthy cell.

As described herein, a mammal (e.g., a human) having OLP can be identified as having an OLP that is likely to proceed to OSCC by determining that cells of the OLP have elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, elevated expression of SRPRB, or a combination thereof. For example, a mammal (e.g., a human) having OLP can be identified as having an OLP that is likely to proceed to OSCC by determining that cells of the OLP have elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB. In some cases, a mammal (e.g., a human) having OLP can be identified as having an OLP that is likely to proceed to OSCC by determining that cells of the OLP have elevated expression of CA1 and elevated expression of TNNT3. In some cases, a mammal (e.g., a human) having OLP can be identified as having an OLP that is likely to proceed to OSCC by determining that cells of the OLP have elevated expression of CA1 and elevated expression of SYNM. In some cases, a mammal (e.g., a human) having OLP can be identified as having an OLP that is likely to proceed to OSCC by determining that cells of the OLP have elevated expression of CA1 and elevated expression of MB. In some cases, a mammal (e.g., a human) having OLP can be identified as having an OLP that is likely to proceed to OSCC by determining that cells of the OLP have elevated expression of CA1 and elevated expression of MYH4. In some cases, a mammal (e.g., a human) having OLP can be identified as having an OLP that is likely to proceed to OSCC by determining that cells of the OLP have elevated expression of CA1 and elevated expression of ST13P4. In some cases, a mammal (e.g., a human) having OLP can be identified as having an OLP that is likely to proceed to OSCC by determining that cells of the OLP have elevated expression of CA1 and elevated expression of MYLPF. In some cases, a mammal (e.g., a human) having OLP can be identified as having an OLP that is likely to proceed to OSCC by determining that cells of the OLP have elevated expression of CA1 and elevated expression of MYH1. In some cases, a mammal (e.g., a human) having OLP can be identified as having an OLP that is likely to proceed to OSCC by determining that cells of the OLP have elevated expression of CA1 and elevated expression of TNNC2. In some cases, a mammal (e.g., a human) having OLP can be identified as having an OLP that is likely to proceed to OSCC by determining that cells of the OLP have elevated expression of CA1 and elevated expression of SRPRB.

In some cases, a mammal (e.g., a human) having OLP can be identified as having an OLP that is likely to proceed to OSCC by determining that cells of the OLP have elevated expression of TNNT3 and elevated expression of SYNM. In some cases, a mammal (e.g., a human) having OLP can be identified as having an OLP that is likely to proceed to OSCC by determining that cells of the OLP have elevated expression of TNNT3 and elevated expression of MB. In some cases, a mammal (e.g., a human) having OLP can be identified as having an OLP that is likely to proceed to OSCC by determining that cells of the OLP have elevated expression of TNNT3 and elevated expression of MYH4. In some cases, a mammal (e.g., a human) having OLP can be identified as having an OLP that is likely to proceed to OSCC by determining that cells of the OLP have elevated expression of a TNNT3 polypeptide and elevated expression of ST13P4. In some cases, a mammal (e.g., a human) having OLP can be identified as having an OLP that is likely to proceed to OSCC by determining that cells of the OLP have elevated expression of TNNT3 and elevated expression of MYLPF. In some cases, a mammal (e.g., a human) having OLP can be identified as having an OLP that is likely to proceed to OSCC by determining that cells of the OLP have elevated expression of TNNT3 and elevated expression of MYH1. In some cases, a mammal (e.g., a human) having OLP can be identified as having an OLP that is likely to proceed to OSCC by determining that cells of the OLP have elevated expression of TNNT3 and elevated expression of TNNC2. In some cases, a mammal (e.g., a human) having OLP can be identified as having an OLP that is likely to proceed to OSCC by determining that cells of the OLP have elevated expression of TNNT3 and elevated expression of SRPRB.

In some cases, a mammal (e.g., a human) having OLP can be identified as having an OLP that is likely to proceed to OSCC by determining that cells of the OLP have elevated expression of SYNM and elevated expression of MB. In some cases, a mammal (e.g., a human) having OLP can be identified as having an OLP that is likely to proceed to OSCC by determining that cells of the OLP have elevated expression of SYNM and elevated expression of MYH4. In some cases, a mammal (e.g., a human) having OLP can be identified as having an OLP that is likely to proceed to OSCC by determining that cells of the OLP have elevated expression of SYNM and elevated expression of ST13P4. In some cases, a mammal (e.g., a human) having OLP can be identified as having an OLP that is likely to proceed to OSCC by determining that cells of the OLP have elevated expression of SYNM and elevated expression of MYLPF. In some cases, a mammal (e.g., a human) having OLP can be identified as having an OLP that is likely to proceed to OSCC by determining that cells of the OLP have elevated expression of SYNM and elevated expression of MYH1. In some cases, a mammal (e.g., a human) having OLP can be identified as having an OLP that is likely to proceed to OSCC by determining that cells of the OLP have elevated expression of SYNM and elevated expression of TNNC2. In some cases, a mammal (e.g., a human) having OLP can be identified as having an OLP that is likely to proceed to OSCC by determining that cells of the OLP have elevated expression of SYNM and elevated expression of SRPRB.

In some cases, a mammal (e.g., a human) having OLP can be identified as having an OLP that is likely to proceed to OSCC by determining that cells of the OLP have elevated expression of MB and elevated expression of MYH4. In some cases, a mammal (e.g., a human) having OLP can be identified as having an OLP that is likely to proceed to OSCC by determining that cells of the OLP have elevated expression of MB and elevated expression of ST13P4. In some cases, a mammal (e.g., a human) having OLP can be identified as having an OLP that is likely to proceed to OSCC by determining that cells of the OLP have elevated expression of MB and elevated expression of MYLPF. In some cases, a mammal (e.g., a human) having OLP can be identified as having an OLP that is likely to proceed to OSCC by determining that cells of the OLP have elevated expression of MB and elevated expression of MYH1. In some cases, a mammal (e.g., a human) having OLP can be identified as having an OLP that is likely to proceed to OSCC by determining that cells of the OLP have elevated expression of MB and elevated expression of TNNC2. In some cases, a mammal (e.g., a human) having OLP can be identified as having an OLP that is likely to proceed to OSCC by determining that cells of the OLP have elevated expression of MB and elevated expression of SRPRB.

In some cases, a mammal (e.g., a human) having OLP can be identified as having an OLP that is likely to proceed to OSCC by determining that cells of the OLP have elevated expression of MYH4 and elevated expression of ST13P4. In some cases, a mammal (e.g., a human) having OLP can be identified as having an OLP that is likely to proceed to OSCC by determining that cells of the OLP have elevated expression of MYH4 and elevated expression of MYLPF. In some cases, a mammal (e.g., a human) having OLP can be identified as having an OLP that is likely to proceed to OSCC by determining that cells of the OLP have elevated expression of MYH4 and elevated expression of MYH1. In some cases, a mammal (e.g., a human) having OLP can be identified as having an OLP that is likely to proceed to OSCC by determining that cells of the OLP have elevated expression of MYH4 and elevated expression of TNNC2. In some cases, a mammal (e.g., a human) having OLP can be identified as having an OLP that is likely to proceed to OSCC by determining that cells of the OLP have elevated expression of MYH4 and elevated expression of SRPRB.

In some cases, a mammal (e.g., a human) having OLP can be identified as having an OLP that is likely to proceed to OSCC by determining that cells of the OLP have elevated expression of ST13P4 and elevated expression ofMYLPF. In some cases, a mammal (e.g., a human) having OLP can be identified as having an OLP that is likely to proceed to OSCC by determining that cells of the OLP have elevated expression of ST13P4 and elevated expression of MYH1. In some cases, a mammal (e.g., a human) having OLP can be identified as having an OLP that is likely to proceed to OSCC by determining that cells of the OLP have elevated expression of ST13P4 and elevated expression of TNNC2. In some cases, a mammal (e.g., a human) having OLP can be identified as having an OLP that is likely to proceed to OSCC by determining that cells of the OLP have elevated expression of ST13P4 and elevated expression of SRPRB.

In some cases, a mammal (e.g., a human) having OLP can be identified as having an OLP that is likely to proceed to OSCC by determining that cells of the OLP have elevated expression of MYLPF and elevated expression of MYH1. In some cases, a mammal (e.g., a human) having OLP can be identified as having an OLP that is likely to proceed to OSCC by determining that cells of the OLP have elevated expression of MYLPF and elevated expression of TNNC2. In some cases, a mammal (e.g., a human) having OLP can be identified as having an OLP that is likely to proceed to OSCC by determining that cells of the OLP have elevated expression of MYLPF and elevated expression of SRPRB.

In some cases, a mammal (e.g., a human) having OLP can be identified as having an OLP that is likely to proceed to OSCC by determining that cells of the OLP have elevated expression of MYH1 and elevated expression of TNNC2. In some cases, a mammal (e.g., a human) having OLP can be identified as having an OLP that is likely to proceed to OSCC by determining that cells of the OLP have elevated expression of MYH1 and elevated expression of SRPRB.

In some cases, a mammal (e.g., a human) having OLP can be identified as having an OLP that is likely to proceed to OSCC by determining that cells of the OLP have elevated expression of TNNC2 and elevated expression of SRPRB.

In some cases, a mammal (e.g., a human) having OLP can be identified as having an OLP that is likely to proceed to OSCC by determining that cells of the OLP have elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, and elevated expression of SRPRB.

Once a mammal (e.g., a human) is identified as having cells containing an elevated level of CA1, an elevated level of TNNT3, an elevated level of SYNM, an elevated level of MB, an elevated level of MYH4, an elevated level of ST13P4, an elevated level of MYLPF, an elevated level of MYH1, an elevated level of TNNC2, an elevated level of SRPRB, or a combination thereof, the mammal can be classified as having OLP that is likely to proceed to OSCC. For example, a human identified as having cells (e.g., oral squamous epithelial or stromal cells affected by OLP) having an elevated level of a CA1 polypeptide or mRNA, an elevated level of a TNNT3 polypeptide or mRNA, an elevated level of a SYNM polypeptide or mRNA, an elevated level of a MB polypeptide or mRNA, an elevated level of a MYH4 polypeptide or mRNA, an elevated level of a ST13P4 mRNA, an elevated level of a MYLPF polypeptide or mRNA, an elevated level of a MYH1 polypeptide or mRNA, an elevated level of a TNNC2 polypeptide or mRNA, an elevated level of a SRPRB polypeptide or mRNA, or a combination thereof can be classified as having OLP that is likely to proceed to OSCC.

In some cases, once a mammal (e.g., a human) is identified as having cells that lack elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, and elevated expression of SRPRB, the mammal can be classified as having OLP that is likely to follow a benign course of the disorder. For example, a human identified as having cells (e.g., oral squamous epithelial or stromal cells affected by OLP) lacking elevated expression of CA1, elevated expression of TNNT3, elevated expression of SYNM, elevated expression of MB, elevated expression of MYH4, elevated expression of ST13P4, elevated expression ofMYLPF, elevated expression of MYH1, elevated expression of TNNC2, and elevated expression of SRPRB can be classified as having OLP that is likely to follow a benign course of the disorder.

As described herein, this document also provides methods and materials for treating a mammal identified as having OLP that is likely to proceed to OSCC. For example, a mammal (e.g., a human) identified as described herein as having OLP that is likely to proceed to OSCC can be administered one or more a topical or systemic immunosuppressive or immunomodulatory agents (e.g., a corticosteroid, dapsone, azathioprine, tacrolimus, mycophenolate mofetil, or a biologic agent). In some cases, a mammal (e.g., a human) identified as described herein as having OLP that is likely to proceed to OSCC can be administered tailored therapies to achieve disease control while minimizing immunosuppression. Having the ability to use more aggressive systemic agents (e.g., azathioprine, tacrolimus, mycophenolate mofetil, or biologic agents) to achieve better disease control can allow clinicians and patients to proceed with treatment options that mitigate the risk for malignant transformation. In some cases, a mammal (e.g., a human) identified as described herein as having OLP that is likely to proceed to OSCC can undergo more regular surveillance (e.g., examination and/or biopsy of involved tissue on a more frequent basis) to detect changes of possible OSCC development early in its course.

In some cases, one or more immunosuppressive or immunomodulatory agents (e.g., a corticosteroid, dapsone, azathioprine, tacrolimus, mycophenolate mofetil, or a biologic agent) can be administered to a mammal once or multiple times over a period of time ranging from days to months. In some cases, one or more immunosuppressive or immunomodulatory agents (e.g., a corticosteroid, dapsone, azathioprine, tacrolimus, mycophenolate mofetil, or a biologic agent) can be formulated into a pharmaceutically acceptable composition for administration to a mammal (e.g., a human) identified as described herein as having OLP that is likely to proceed to OSCC. For example, a therapeutically effective amount of an immunosuppressive or immunomodulatory agent (e.g., a corticosteroid, dapsone, azathioprine, tacrolimus, mycophenolate mofetil, or a biologic agent) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more immunosuppressive or immunomodulatory agents (e.g., a corticosteroid, dapsone, azathioprine, tacrolimus, mycophenolate mofetil, or a biologic agent) can be designed for oral, topical, or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition containing one or more immunosuppressive or immunomodulatory agents (e.g., a corticosteroid, dapsone, azathioprine, tacrolimus, mycophenolate mofetil, or a biologic agent) can be administered locally or systemically. For example, a composition provided herein can be administered locally by intravenous injection or blood infusion. In some cases, a composition provided herein can be administered systemically, orally, or by injection to a mammal (e.g., a human).

Effective doses can vary depending on the severity of the OLP, the route of administration, the age and general health condition of the subject, excipient usage, the possibility of co-usage with other therapeutic treatments, and the judgment of the treating physician.

An effective amount of a composition containing an immunosuppressive or immunomodulatory agent (e.g., a corticosteroid, dapsone, azathioprine, tacrolimus, mycophenolate mofetil, or a biologic agent) can be any amount that reduces the likelihood that the OLP will progress to OSCC without producing significant toxicity to the mammal. For example, an effective amount of an immunosuppressive or immunomodulatory agent (e.g., a corticosteroid, dapsone, azathioprine, tacrolimus, mycophenolate mofetil, or a biologic agent) can be from about 0.25 mg/kg to about 100 mg/kg (e.g., from about 0.3 mg/kg to about 11 mg/kg, from about 1 mg/kg to about 10 mg/kg, from about 2 mg/kg to about 10 mg/kg, from about 5 mg/kg to about 10 mg/kg, from about 6 mg/kg to about 10 mg/kg, from about 6 mg/kg to about 8 mg/kg, or from about 7 mg/kg to about 9 mg/kg). In some cases, from about 100 mg to about 1000 mg (e.g., from about 250 mg to about 1000 mg, from about 300 mg to about 1000 mg, from about 400 mg to about 1000 mg, from about 100 mg to about 900 mg, from about 100 mg to about 800 mg, from about 400 mg to about 800 mg, or from about 500 mg to about 700 mg) of an immunosuppressive or immunomodulatory agent (e.g., a corticosteroid, dapsone, azathioprine, tacrolimus, mycophenolate mofetil, or a biologic agent) can be administered to an average sized human (e.g., about 75-85 kg human) per administration (e.g., per daily or weekly administration) for about two to about twelve weeks or more.

If a particular mammal fails to respond to a particular amount, then the amount of the administered immunosuppressive or immunomodulatory agent(s) can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of an immunosuppressive or immunomodulatory agent (e.g., a corticosteroid, dapsone, azathioprine, tacrolimus, mycophenolate mofetil, or a biologic agent) can be any frequency that reduces the likelihood that the OLP will progress to OSCC without producing significant toxicity to the mammal. For example, the frequency of administration can be from about once a day to about once a month (e.g., from about once a week to about once every other week). The frequency of administration can remain constant or can be variable during the duration of treatment. A course of treatment with a composition containing one or more immunosuppressive or immunomodulatory agents (e.g., a corticosteroid, dapsone, azathioprine, tacrolimus, mycophenolate mofetil, or a biologic agent) can include rest periods. For example, a composition containing an immunosuppressive or immunomodulatory agent (e.g., a corticosteroid, dapsone, azathioprine, tacrolimus, mycophenolate mofetil, or a biologic agent) can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the OLP may require an increase or decrease in administration frequency.

An effective duration for administering a composition containing one or more immunosuppressive or immunomodulatory agents (e.g., a corticosteroid, dapsone, azathioprine, tacrolimus, mycophenolate mofetil, or a biologic agent) can be any duration that reduces the likelihood that the OLP will progress to OSCC without producing significant toxicity to the mammal. In some cases, the effective duration can vary from several days to several months. In general, the effective duration for reducing the likelihood that OLP will progress to OSCC can range from about six weeks to about six months. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated (e.g., OLP) can be monitored. Any appropriate method can be used to determine whether or not a mammal's likelihood of developing OSCC is being delayed or reduced. For example, cells can be assessed following administration of an immunosuppressive or immunomodulatory agent (e.g., a corticosteroid, dapsone, azathioprine, tacrolimus, mycophenolate mofetil, or a biologic agent) to determine a lack of progression to OSCC as occurred.

The invention will be further described in the following examples, which do not limit the scope of the invention described in the claims.

### EXAMPLES

### Example 1—Whole-Exome Sequencing of Transforming Oral Lichen Planus Reveals Mutations in DNA Damage Repair and Apoptosis Pathway Genes

### Materials and Methods

### Patient Selection

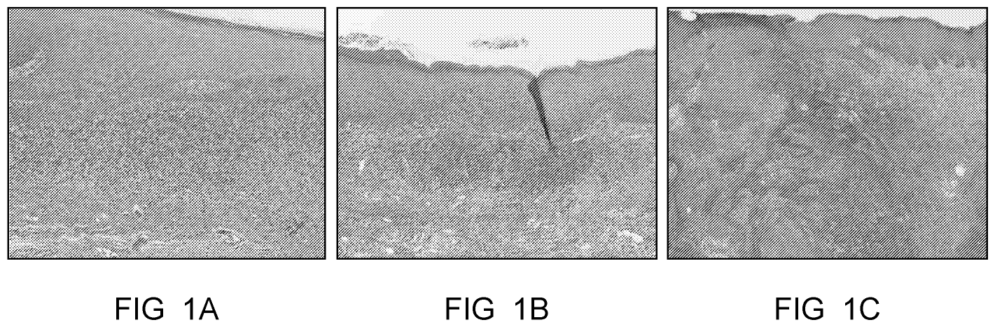

A retrospective review was performed of the electronic medical record for patients with a clinical diagnosis of oral lichen planus (OLP). Clinical diagnoses were made by board-certified dermatologist or oral medicine specialist. All diagnoses were verified by a board-certified dermatopathologist based on modified WHO criteria (van der Meij and van der Waals, J. Oral Pathol. Med., 32:507-512 (2003)) and the American Academy of Oral and Maxillofacial Pathology criteria (Cheng et al., Oral Surg. Oral Med. Oral Pathol. Oral Radiol., 122:332-354 (2016)). Specifically, OLP diagnosis required absent squamous dysplasia and absent verrucous epithelial architectural change (FIGS. 1A-1C). Screening for high-risk HPV infection was accomplished with p16 immunohistochemical staining, with >70% (block) epithelial staining counting as positive. Representative areas of OLP and oral squamous cell carcinoma (OSCC) were obtained from micro-dissected FFPE tissues, either manually or with laser capture microdissection, depending on the nature of the specimen.

### DNA Extraction

DNA was isolated from FFPE tissue using QIAamp DNA FFPE Tissue Kit (Qiagen, Hilden, Germany) according to manufacturer's instructions. Briefly, 10 m sections were deparaffinized by dissolving in xylene, then lysed under denaturing conditions with proteinase K, and incubated at 90° C. to reverse formalin crosslinking. Residual contaminants were washed away to elute pure, concentrated DNA and stored at −80° C. until use.

### Exome Sequencing

Paired-end libraries were prepared from as low as 30 ng to about 300 ng of FFPE DNA using the SureSelect XT HS Reagent Kit (Agilent, Santa Clara, CA). The concentration and size distribution of the completed libraries were determined using an Agilent Bioanalyzer DNA 1000 chip or Advance Fragment Analyzer and Qubit fluorometry (Invitrogen, Carlsbad, CA). Whole exon capture was carried out using 750 ng of the prepped library following the protocol for Agilent's SureSelect Human All Exon v5+UTRs 75 MB kit. The purified capture products were then amplified for 12 cycles. The concentration and size distribution of the completed captured libraries were determined by Qubit and the Agilent Bioanalyzer DNA 1000 chip. Libraries were sequenced at six samples per lane following Illumina's standard protocol using the Illumina cBot and HiSeq 3000/4000 PE Cluster Kit. The flow cells were sequenced as 150×2 paired end reads on an Illumina HiSeq 4000 using the HiSeq 3000/4000 sequencing kit and HD 3.4.0.38 collection software. Base-calling was performed using Illumina's RTA version 2.7.7.

### Somatic Variant Calling and Filtering

An ensemble variant calling approach was utilized for identifying likely somatic variants in the samples. This process started by analyzing the quality of the reads produced by each sample using FASTQC software (version 0.11.8), and adapters were trimmed using trimgalore software (version 0.4.4). Processed reads were aligned to human genome (GRCh38) using bwa software (version 0.7.17). Variants were called using four different callers: Mutect2 (version GATK 4.1.8.1), Pisces (version 5.2.10.49), FreeBayes (version 1.3.1), and LoFreq (version 2.2.1). Variants detected by at least three independent callers were selected for further analysis.

An in-house script in R programming language was developed to combine the variant calls from all samples and filter them to determine likely somatic variants. First, a variant quality filter that dropped variants with allele frequency of <10%, total read depth of <10 and alternate read depth of <2 was instituted. Next, variants associated with FFPE fixation process were removed. This was accomplished by processing N=8 normal tonsil FFPE samples (part of another study not described here) using the same WES and bioinformatics protocol described above and removing any variants in the LP cohort that were also observed in the normal tonsil FFPE cohort. Next, potential polymorphisms using gnomAD (population frequency>0.01%) and Mayo Clinic normal whole exome samples (presence as filter) were removed. Variants that were in low complexity regions were also removed. Finally, a backfill strategy was instituted to recover any low allele frequency variants that are likely pathogenic. This was accomplished by taking variants with an allele frequency between 5%-10% and filtering using the above described strategy. A variant in this list was added to the cohort if it was detected in at least one other sample with allele frequency>10%. Finally, a gene-level recurrence filter of >=2 samples was applied to extract recurrent variants.

The target region was 75 Mb in each sample, and the sequence coverage was 40× on average. A total of 5451 likely somatic variants were called after filtering. This included 122 deletions, 109 insertions, 4503 non-synonymous mutations, and 114 frameshift mutations.

### Mutational Signatures and Therapeutic Targets

MutSignatures software (version 2.1.3) was used to decipher the presence of known tumor mutation signatures that were described elsewhere (Alexandrov et al., Cell Rep., 3:246-259 (2013)). For this, variants that passes the above described filtration algorithm with exception of gene-level recurrence filter were selected. The software was instructed to deconvolute the mutational profile of each sample into known tumor mutation signatures and report the percent contribution of each signature in each sample. A Student's t-test was performed to detect signatures that were enriched in transforming OLP or OSCC samples when compared to indolent OLP samples. Signatures that had a p-value<=0.05 in at least one of the comparisons were considered as significant for reporting.

### Statistical Analysis

A two-sided false discovery rate (FDR) adjusted p-value of <0.05 was considered significant. Statistical analysis was performed using JMP Pro statistical software version 14.1 (SAS Institute, Cary, NC).

### Results

### Patient Characteristics

17 patients with indolent OLP, 9 patients with transforming OLP, and 17 patients with OSCC arising from areas of OLP were included. Samples from these patients included 8 pairs of OSCC with adjacent transforming OLP tissue. Patient characteristics are summarized in TABLE 1.

TABLE 1
Patient characteristics, by disease category.

| | | Transforming OLP | OSCC |
|---|---|---|---|
| | Indolent OLP | n = 9 patients | n = 17 patients |
| | n = 17 patients | (9 samples; | (17 samples; |
| | (18 samples) | 8 paired) | 8 paired) |
| Mean age (years; range) | 61 (50-73) | 66 (23-83) | 65 (23-83) |
| Sex | 8 (47%) male: 9 | 4 (44%) male: 5 | 7 (41%) male: 10 |
| | (53%) female | (56%) female | (59%) female |
| Anatomic site of biopsy | 12 (71%) buccal: 3 | 5 (56%) tongue: | 9 (53%) tongue: 4 |
| | (18%) gingival: 2 | 3 (33%) gingival: | (24%) buccal: 4 |
| | (12%) tongue | 1 (11%) lip | (24%) gingival |
| Substantial tobacco | 9 (53%) past use: 1 | 3 (33%) past use | 3 (18%) past use: 1 |
| use (>5 pack years) | (6%) current use | | (6%) current use |
| Long-term substantial | 1 current (6%): 1 | 1 (11%) current | 1 (6%) current |
| alcohol (>14 units/week) | (6%) prior | | |
| Oral high-risk HPV | 0 | 0 | 0 |
| infection (as implied by p16 | | | |
| immunohistochemistry | | | |
| positivity) | | | |
| Hepatitis C infection | 1 (6%) | 0 | 0 |
| (clinical) | | | |
| Clinical follow up, mean | 14 (6-25) | 4 (0.33-6) | 4 (0.25-16) |
| (years; range) | | | |
| OLP subtype | 5 (29%) reticular: 5 | 4 (44%) erosive: | 10 (59%) erosive: |
| | (29%) erosive: 1 | 2 (22%) reticular: | 1 (6%) reticular: 1 |
| | (6%) atrophic: 6 | 1 (11%) plaque: | (6%) plaque: 4 |
| | (35%) unclear | 2 (22%) unclear | (24%) unclear |
| OSCC microscopic subtype | N/A | N/A | 1 (6%) carcinoma |
| | | | in situ: 2 (12%) |
| | | | well differentiated: |
| | | | 13 (76%) |
| | | | moderately |
| | | | differentiated: 1 |
| | | | (6%) poorly |
| | | | differentiated |
| Deceased at the time of last | 1 (6%; unrelated) | 5 (56%; 3 from | 10 (59%; 7 from |
| follow-up (cause, in relation | | OSCC, 1 | OSCC, 1 |
| to OSCC) | | unrelated) | unrelated) |
| OSCC metastasis | N/A | 3 (33%) | 6 (35%) |
| Dentures | 2 (12%) | 2 (22%) | 5 (29%) |
| OLP treatment, prior to | 4 (24%) | 4 (44%) | 13 (76%) |
| biopsy | | | |
| Duration of disease until | 29 (1-84) | 71 (6-240) | 76 (12-240) |
| biopsy, mean (months; range) | | | |

OLP, Oral lichen planus;
OSCC, oral squamous cell carcinoma;
HPV, human papillomavirus;
N/A, not applicable

### Profile of Mutations in OSCC

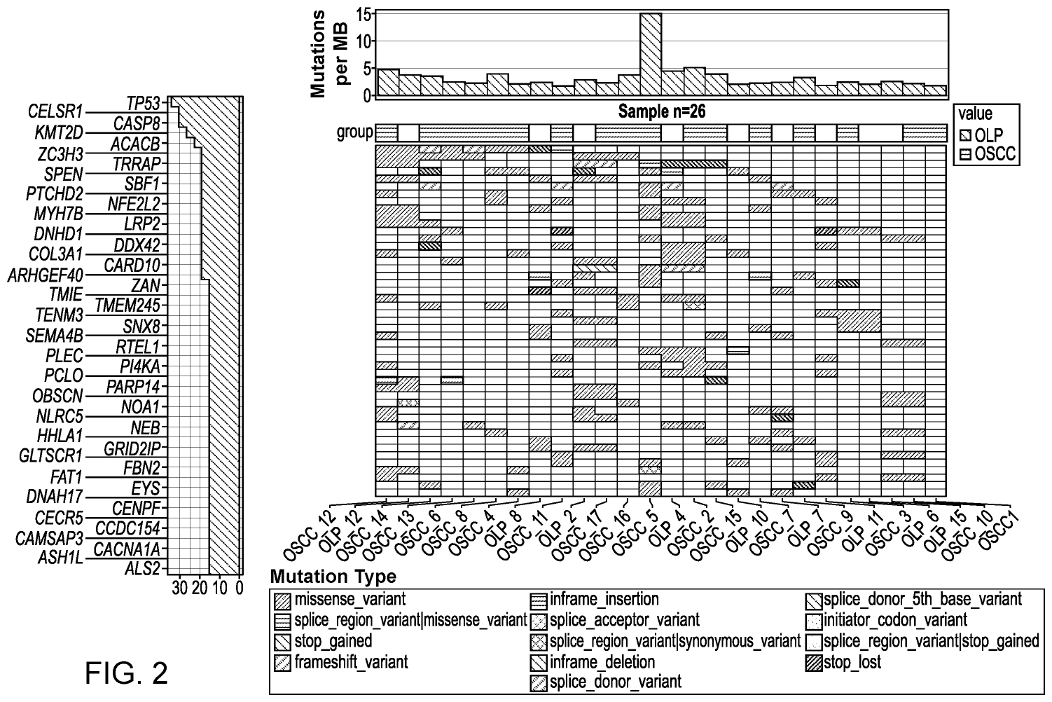

TP53 was the most frequently mutated gene (7/17 samples; 41%) in OSCC samples, followed by CELSR1 (6/17 samples; 35%), and then PVRL1, POU4F2 and CASP8 (5/17 samples; 29%; FIG. 2). TP53, CELSR1, and KMT2D were shared between transforming OLP and OSCC, OBSCN between indolent OLP and OSCC, and PVRL1 and POU4F2 were also expressed in both indolent and transforming OLP.

### Differences Between Indolent and Transforming OLP

Mutations present in transforming OLP but absent in indolent OLP were considered to be possible driver genes for carcinogenesis. The most frequently mutated genes in transforming but not indolent OLP were TMIE, KMT2D, HFM1, CENPF, and ACACB (FIG. 2). NOTCH4 and CDCP2 were common to both indolent and transforming OLP.

Profile of Mutations in Transforming OLP and OSCC that are not in Indolent OLP The variants seen in transforming OLP and OSCC (conditions with malignant outcome) but not in indolent OLP, in those with 10% variance, were plotted, and then those genes were backfilled to include any samples seen in <10% variance in any other samples (FIG. 2). Mutations were generally low (<6 mutations) except for in one OSCC sample (OSCC_5) containing 15 mutations. This sample was derived from a 57 year-old-man with 40 pack-year smoking history and nodal OSCC metastases.

The four most frequently mutated genes in transforming OLP and OSCC were selected: TP53, CELSR1, CASP8, KMT2D. See, e.g., TABLE 3. Using this panel of genes, 12/17 (71%) of OSCC samples and 5/9 (55%) of transforming OLP samples were successfully predicted. These genes were not mutated in indolent OLP.

### Paired Samples

The exact mutation profile in the 8 paired samples of OSCC and adjacent transforming OLP tissue was compared (TABLE 2). When mutations of the same gene were present in both pairs, the nucleotide change and location were uniformly identical.

TABLE 2
Paired samples to check for exact mutation present in oral squamous
cell carcinoma (OSCC) and transforming oral lichen planus (OLP).

| | Present in | Present in | Present in indolent |
|---|---|---|---|
| Genes | both pairs | OSCC only | OLP only |
| TP53 | 1 | 2 | 1 |
| CELSR1 | 2 | | |
| KMT2D | 1 | | 2 |
| CASP8 | 3\hat{ } | | |
| OBSCN | | | |
| TTRAP | | 1 | |
| LRP2 | 1 | | |
| TENM3 | | 1 | |
| ASH1L | | | |
| FAT1 | 1* | | 1 |

*One pair had 3 FAT1 mutations in both pairs
\hat{ }One pair had 2 CASP8 mutations in both pairs.

TABLE 3
Mutations detected in nucleic acid encoding TP53, CELSR1,
CASP8, and KMT2D polypeptides.

| Gene | Assignment ID | Codon change |
|---|---|---|
| TP53 | ENST00000618944 | c.413dupA |
| | | c.266G > A_p.Arg89Gln |
| | | c.265C > T_p.Arg89Trp |
| | | c.234G > A_p.Met78Ile |
| | | c.195 + 2T > C |
| | | c.47G > A_p.Arg16His |
| | | c.47G > A_p.Arg16His |
| | | c.47G > A_p.Arg16His |
| | ENST00000615910 | c.309C > A_p.Tyr103X |
| | | c.138_154del17 |
| CASP8 | ENST00000392266 | c.319C > T_p.Gln107X |
| | | c.319C > T_p.Gln107X |
| | | c.757G > A_p.Gly253Arg |
| | ENST00000264274 | c.776dupT |
| | | c.776dupT |
| | | c.820C > T_p.Gln274X |
| | | c.820C > T_p.Gln274X |
| | | c.829C > T_p.Gln277X |
| | | c.928C > T_p.Pro310Ser |
| | | c.928C > T_p.Pro310Ser |
| CELSR1 | ENST00000262738 | c.8023G > A_p.Val2675Met |
| | | c.7996G > A_p.Ala2666Thr |
| | | c.6653G > A_p.Arg2218Gln |
| | | c.5179G > A_p.Val1727Ile |
| | | c.4108G > A_p.Asp1370Asn |
| | | c.3629G > A_p.Arg1210His |
| | | c.3629G > A_p.Arg1210His |
| | | c.2296G > A_p.Asp766Asn |
| | | c.1307C > T_p.Pro436Leu |
| | | c.1307C > T_p.Pro436Leu |
| KMT2D | ENST00000301067 | c.11737C > T_p.Gln3913X |
| | | c.8048G > A_p.Arg2683His |
| | | c.6464C > T_p.Ser2155Leu |
| | | c.6001C > T_p.Arg2001Trp |
| | | c.2992C > G_p.Pro998Ala |
| | | c.2992C > G_p.Pro998Ala |
| | | c.1010C > A_p.Ser337X |

### Profile of Mutations in Indolent OLP

Mutations were found in 16 genes that were mutated in at least 20% of tested samples (≥4/18). Most frequently mutated genes were SYNE1, RYR1, PVRL1, NOTCH4, NOP9, FAM83D and CCDC168.

### Mutational Profile in Metastasizing OSCC

Six of 17 (35%) OSCC samples were derived from patients who developed metastases. After removing mutations found in the indolent OLP group, no significant differences were found in mutations between OSCCs that did not metastasize versus OSCCs with metastases (Chi-squared, p=0.9955).

### Mutational Signatures

Mutational signatures in transforming OLP and OSCC that were not present in indolent OLP were considered those associated with malignant risk. It was discovered that, at the individual sample level, the single-base substitutions COSMIC SBS3 DNA double-strand break repair and SBS26 DNA mismatch repair were significantly enriched.

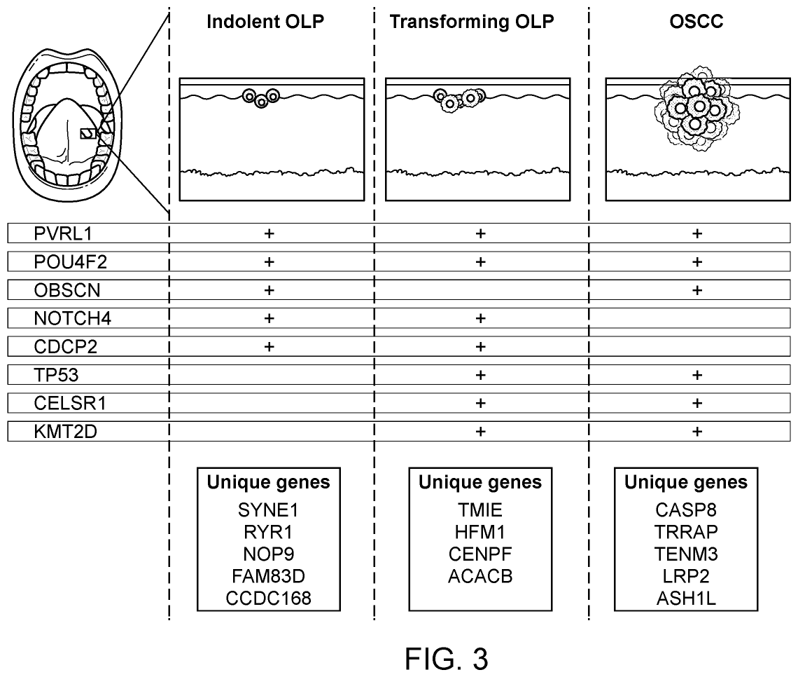

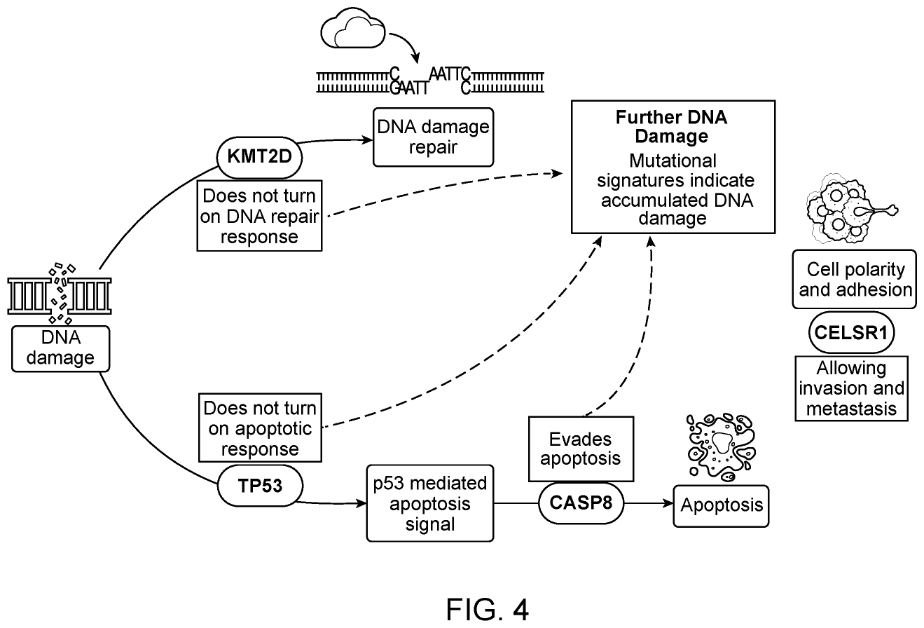

These results demonstrate the discovery of shared somatic mutations in transforming OLP and OSCC (FIG. 3), with the most frequent shared driver mutations being TP53, KMT2D, CELSR1 and CASP8. Based on these findings and the biologic function of these genes, a potential pathway for carcinogenesis from OLP to OSCC can be as shown in FIG. 4. Enrichment of mutational signatures indicating accumulated DNA damage further supports the altered response to DNA damage. Taken together, these results highlight the premalignant nature of OLP with the presence of known cancer driver mutations in transforming OLP that may enable further carcinogenesis by altering DNA damage repair and apoptosis pathways.

By identifying samples from OLP specifically, mutations specific to OLP-associated OSCC were identified. An unbiased approach was used by sequencing the whole protein coding genome, minimizing bias in comparison to prior experiments focusing one or a few selected genes. Rigorous diagnostic and histopathological criteria for inclusion were applied, and a long follow-up period for indolent OLP with average 14 years (6-25 years) was used.

### Example 2—Differential Proteomic Expression in Indolent Versus Transforming Oral Lichen Planus

### Materials and Methods

### Patient Selection

Three groups of patients were identified through retrospective manual electronic medical record review: a) 6 patients with indolent OLP (no OSCC with at least 5 years of clinical follow-up), b) 6 patients with transforming OLP (non-dysplastic tissue marginal to OSCC, defined as any grade of invasive OSCC in individuals with prior history of OLP), and c) 5 individuals with NOM from amalgam tattoo biopsies (control). There were insufficient OLP samples predating OSCC to be used as controls.

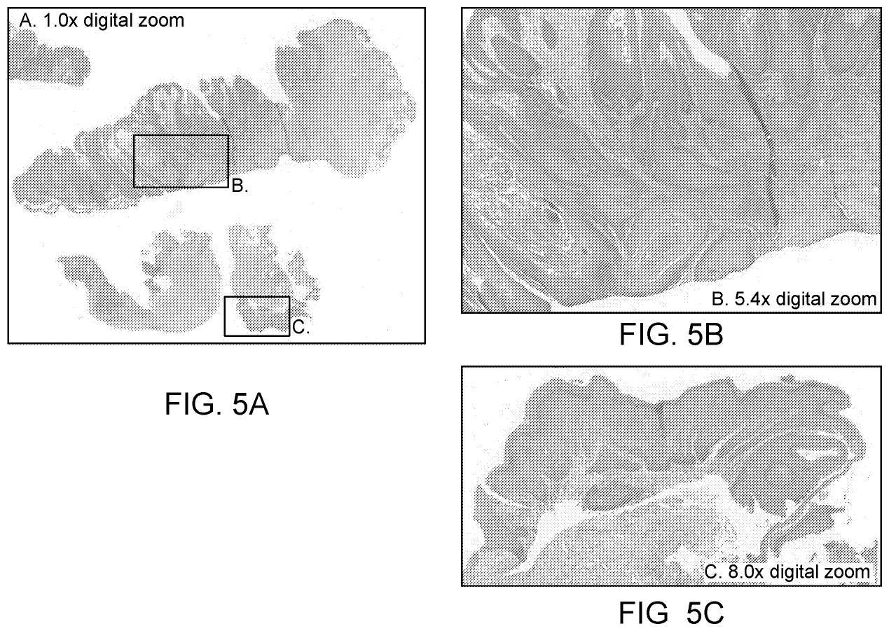

Clinical diagnoses were made by a board-certified dermatologist or oral medicine specialist, and retrospectively verified by a board-certified dermatopathologist. Diagnosis of OLP was based on the modified WHO criteria (van der Meij and van der Waal, supra) and the American Academy of Oral and Maxillofacial Pathology criteria (Cheng et al., supra), which required that there be no dysplasia or verrucous epithelial architectural change. High-risk HPV infection was defined as >70% (block) positive epithelial p16 immunohistochemical staining. Additionally, cases were excluded if there was diagnostic uncertainty, or if insufficient tissue remained in paraffin blocks. Wherever possible, tissue immediately adjacent to the OSCC was avoided to minimize spurious results due to field cancerization effect (FIG. 5). There was no overlying dysplasia.

### Tissue Collection

Formalin-fixed paraffin-embedded (FFPE) oral mucosal biopsies from the aforementioned patients were used for mass spectrometry. Proteins were extracted and subjected to proteomics analysis as described elsewhere (Graham et al., Mod. Pathol., 29(6):607-615 (2016); and Gleue et al., Exp. Dermatol. doi: 10.1111/exd.14660, (2022)). In brief, 10 m-thick sections were obtained for each patient and stained with hematoxylin-and-eosin (H&E). Unstained sections of the same thickness were mounted on polyethylene naphthalate (PEN) membrane slides for laser pressure catapulting to collect tissue (Zeiss Microbeam). H&E-stained sections were utilized as guide slides to identify areas of interest in tissue to be captured on the PEN membrane slides. 1 mm² of specified tissue regions for each patient were collected by laser capture microdissection into digest buffer (100 mM Tris, pH 8.2; 0.005% Zwittergent 3-16) in caps of 0.5 ml tubes. Tubes were spun to collect tissue in the buffer, and all subsequent procedures were done in the same tubes. Formalin bonds in proteins were broken by incubation of samples at 98° C. for one hour, and proteins were subsequently reduced and alkylated with dithiothreitol and iodoacetamide, respectively. Resulting denatured proteins were digested by incubating with trypsin overnight at 37° C.

### Mass Spectrometry

Peptide digests were desalted with C18 tips and reconstituted in 0.1% TFA. About 10 μl of peptide digest from each sample was loaded onto a 0.33 mL Halo2.7 ES-C18 trap. Captured peptides were separated using a 35 cm×100 mM Poroshell reverse phase column (Agilent; Santa Clara, CA) packed with C18 connected to a RS3000 Nano liquid chromatography (LC; Thermo-Fisher, Waltham, MA) system. Eluting peptides were analyzed using a Q EXACTIVE™ (Thermo-Fisher; Waltham, MA) mass spectrometer configured to collect data in data dependent acquisition mode. In this mode, the mass spectrometer was configured to use a MS resolution of 70,000, with an automatic gain control (AGC) of 1e⁶, maximum ion trap (IT) time of 50 ms, and scan range of 340-1500 m/z. The top 15 peptide ions identified in each MS scan were subjected to MS/MS using a resolution of 17,500, with AGC of 1e⁵ (minimum AGC of 4e³), intensity threshold of 8e⁴, maximum IT time of 50 ms, an isolation window of 3.0 m/z, normalized collision energy of 26, and scan range of 200-2000 m/z. Only peptides with charge states of 2-4 were considered for MS/MS analysis. Raw data acquired from all samples were processed to extract the protein identification and quantification information as described below.

### Proteomic and Statistical Analysis

A bioinformatics method described elsewhere (Ayers-Ringler et al., Front. Behav. Neurosci., 10:46 (2016)) was used for processing the raw data files and generating peptide identifications, protein identifications and perform MS1 signal-based label-free quantification. In brief, MaxQuant software (version 1.6.0) was configured to use a composite human protein sequence database containing UniProt human reference proteome (downloaded on Feb. 12, 2018). Reversed protein sequences were appended to the database for estimating protein identification false discovery rates (FDRs). The software filtered peptide and protein identifications at 1% FDR, grouped protein identifications into groups and reported protein group intensities. An in-house script written in R programming language performed differential expression analysis using protein group intensities. First, protein group intensities of each sample were log 2 transformed and normalized using Quantile method. For each protein group, the normalized intensities observed in two groups of samples were modeled using a Gaussian-linked generalized linear model. An ANOVA test was used to detect the differentially expressed protein groups between pairs of experimental groups. Differential expression p-values were FDR corrected using Benjamini-Hochberg-Yekutieli procedure. Protein groups with an FDR<0.05 and an absolute log 2 fold change of at least 1.0 (where 0.0 corresponds to no change) were considered as significantly differentially expressed and saved for pathway analysis.

### Pathway Analysis

Pathway analysis was performed using QIAGEN IPA (QIAGEN Inc., digitalinsights.qiagen.com/IPA) (Kramer et al., Bioinformatics, 30(4):523-530 (2014)) and gene set enrichment analysis, GSEA software, and Molecular Signature Database (MsigDB) (Subramanian et al., Proc. Natl. Acad. Sci. USA, 102(43):15545-15550 (2005); and Mootha et al., Nat. Genet., 34(3):267-273 (2003)) to assess the functional relevance of the observed differences in gene expression profiles.

### Results

### Patient Population

Six cases each of indolent OLP and transforming OLP, along with five cases of NOM were identified (TABLE 4). The patients were similar in age across the 3 groups (67±6.6 in indolent OLP; 65±20.4 in transforming OLP; and 72±11.2 in NOM). The majority of indolent OLP biopsies were taken from buccal mucosa (5/6; 83%), while the majority of transforming OLP biopsies were from the tongue (3/6; 50%). None of the samples showed block positivity for p16, the proxy for high-risk HPV infection. Mean clinical follow up was 11 years (range 6-17 years) for indolent OLP, compared to 6 years (range 0-15 years) for transforming OLP.

### Transforming OLP Vs Indolent OLP

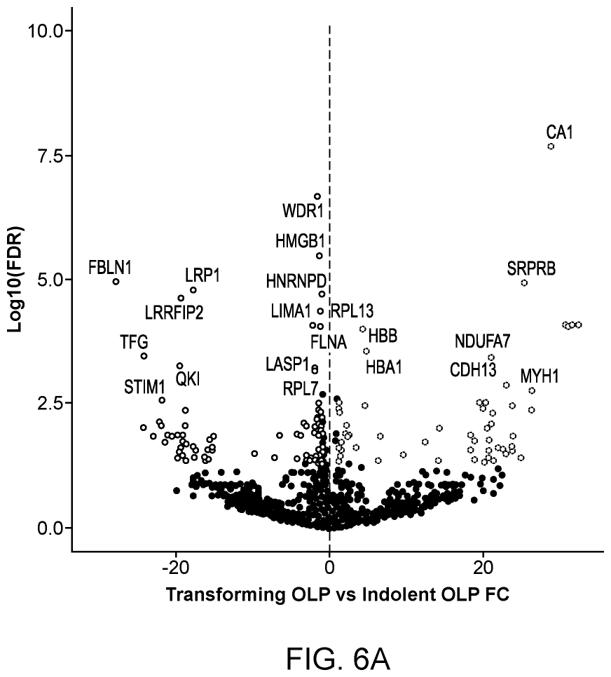

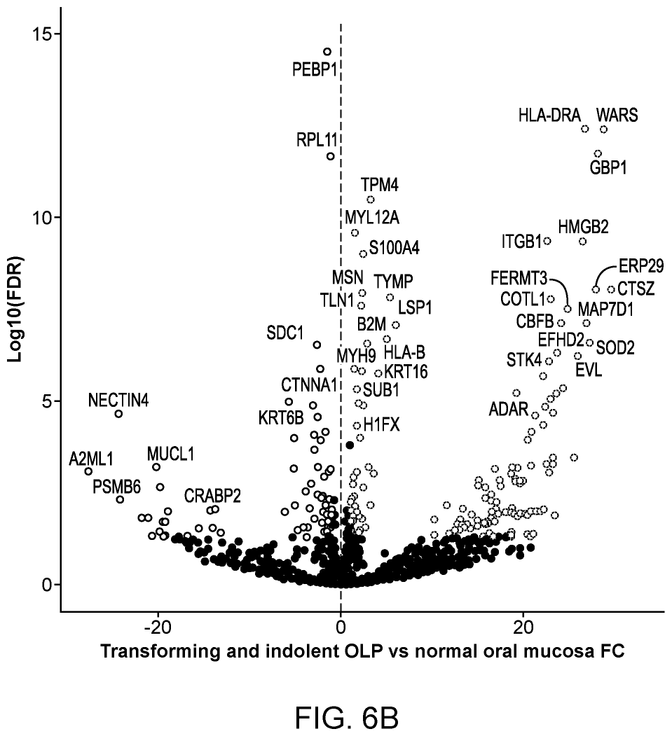

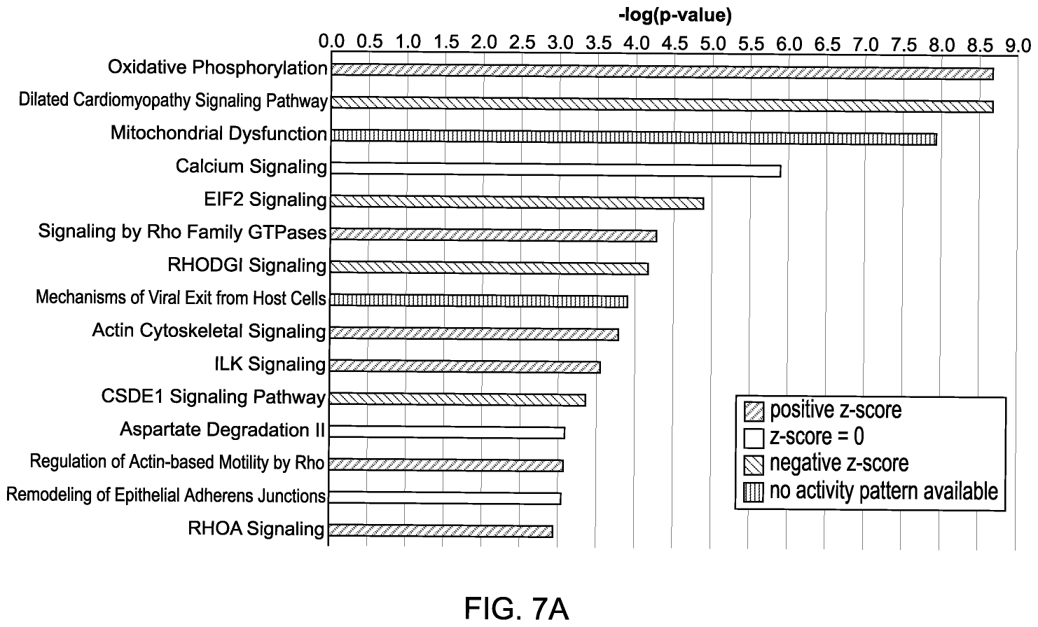

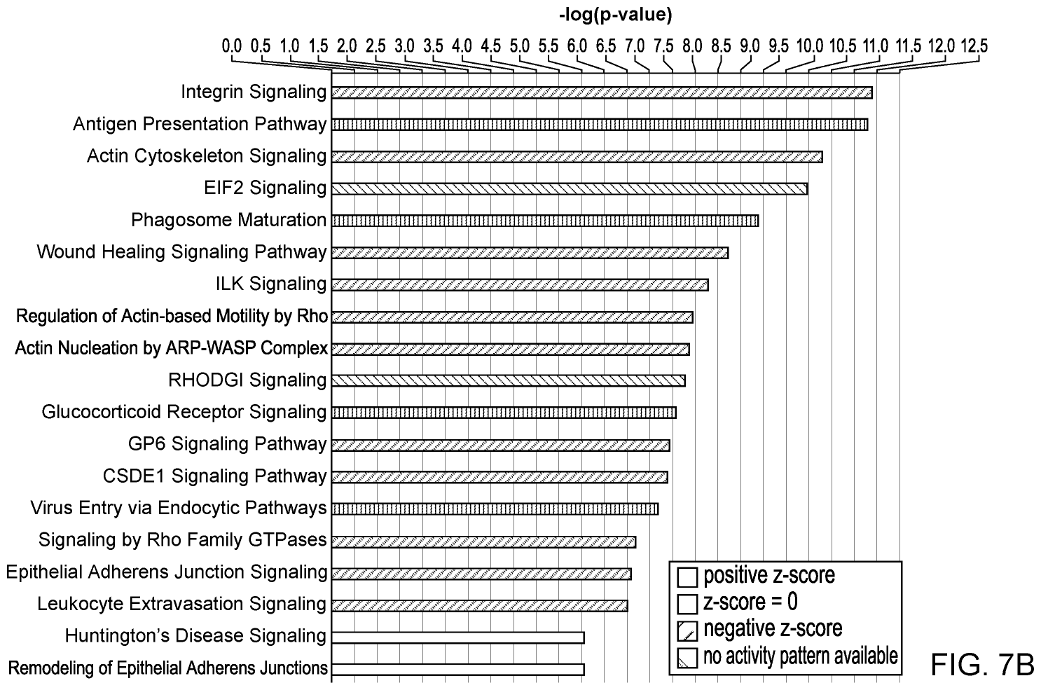

Proteomic analysis of transforming OLP compared to indolent OLP revealed 1431 distinct proteins at false discovery rate of <0.01. 1-fold or more and FDR<0.05 differentially expressed proteins were included in volcano plots (FIG. 6A) and pathway analyses. The most differentially expressed proteins (≥25-fold or ≤25-fold) are shown in TABLE 5. These data represent proteins that have a binary expression profile between the two groups (i.e., present vs. absent). The most overexpressed genes were TNNT3, SYNM, MYH4, ST13P4, MB, MYLPF, CA1, TNNC2, MYH1, SRPRB, and the most underexpressed were FBLN1 and VCAN. IPA highlighted oxidative phosphorylation, calcium signaling, mitochondrial dysfunction, EIF2 signaling, actin cytoskeleton signaling, dilated cardiomyopathy signaling and remodelling of epithelial adherens junctions pathways (FIG. 7A). MYC, MAPT, torin1 and SMTNL1 were key upstream regulators. GSEA enriched pathways revealed overexpressed pathways in oxidative phosphorylation, respiratory electron transport, fatty acid metabolism, and muscle contraction (FDR q-value<0.05). Similar pathways in actin cytoskeleton and adherens junction pathways were enriched when indolent and transforming OLP were compared against NOM. Further, the same pathways was observed to be enriched when comparing transforming OLP against indolent OLP, suggesting that they are likely to be more predominant in the transforming OLP samples. All H&E slides were re-examined to confirm that skeletal muscle was absent from the tissue blocks.

### OLP (Indolent and Transforming) Vs NOM

Proteomic analysis of OLP cases (indolent and transforming) compared to NOM revealed 1447 proteins at false discovery rate of <0.01. Proteins with 1-fold or more and false discovery rate (FDR)<0.05 were included in volcano plots (FIG. 6B) and pathway analyses. Proteins differentially expressed 25-fold or more in indolent and transforming OLP in comparison with NOM are shown in TABLE 6. These data represent proteins that have a binary expression profile between the two groups (i.e., present vs. absent). The most overexpressed proteins included LYZ, TAPBP, ANXA6, ICAM1, CTSZ, LBR, WARS, PTPRCAP, GBP1, ERP29, PTPRC, SOD2, MAP7D1, and HLA-DRA, while A2ML1 was the most underexpressed. Ingenuity pathway analyses (IPA) enriched for canonical pathways related to inflammation and muscle structural pathways, including in integrin signaling, antigen presentation, actin cytoskeleton, regulation of actin-based motility by Rho, glucocorticoid receptor signaling and EIF2 signaling (FIG. 7B). MYC, IL4 and TP53 were upstream regulators.

Taken together, these studies demonstrated that the transforming OLP protein profile was enriched for actin cytoskeleton, mitochondrial dysfunction, and oxidative phosphorylation pathways. CA1, TNNT3, SYNM, MB were overexpressed, and FBLN1 was underexpressed in transforming OLP compared to indolent OLP. Integrin signaling and antigen presentation pathways were enriched in both indolent and transforming OLP compared to NOM. Thus, these proteomic studies provide potential biomarkers, such as CA1 overexpression, for higher risk OLP. The actin cytoskeleton and muscle contraction pathways were enriched in transforming OLP, suggesting that extracellular matrix stability and possibly epithelial-mesenchymal transition (EMT) are important in OLP transformation to OSCC. The differentially expressed proteins in transforming OLP may be leveraged as prognostic biomarkers or therapeutic targets.

TABLE 4
Patient demographics of selected cases of normal oral mucosa (NOM), indolent orallichen
planus (OLP), and transforming OLP.

| | | | Transforming |
|---|---|---|---|
| | NOM | Indolent OLP | OLP |
| | n = 5 | n = 6 | n = 6 |
| Age (years; | 72 ± 11.2 | 67 ± 6.6 | 65 ± 20.4 |
| mean ± SD) | | | |
| Sex | 1 (20%) male; 4 | 3 (50%) male; | 3 (50%) male; 3 |
| | (80%) female | 3 (50%) female | (50%) female |
| Anatomic site of | 2 (40%) buccal; 2 | 5 (83%) | 3 (50%) tongue; 1 |
| biopsy | (40%) floor of | buccal; 1 | (17%) buccal; 1 |
| | mouth; 1 (20%) | (17%) lip | (17%) lip; 1 (17%) |
| | gingiva | | gingiva |
| Substantial tobacco | 2 (40%) past use | 2 (33%) past | 3 (50%) past use; 1 |
| use (>5 pack years) | | use; 1 (17%) | (17%) current use |
| | | current use | |
| Long-term substantial | 0 | 0 | 0 |
| alcohol (>14 | | | |
| units/week) | | | |
| Oral high-risk HPV | 0 | 0* | 0 |
| infection (as implied | | | |
| by p16 | | | |
| immunohistochemistry | | | |
| positivity) | | | |
| Hepatitis C infection | 0 | 0 | 0 |
| Clinical follow up, | 8 (1-17) | 11 (6-17) | 6 (0-15) |
| mean (years; range) | | | |
| OLP subtype | N/A: patients with | 3 (50%) | 2 (33%) erosive; 1 |
| | NOM from | erosive; 1 | (17%) reticular; 1 |
| | amalgam tattoo | (17%) | (17%) plaque; 2 |
| | biopsy | reticular; 1 | (33%) unknown |
| | | (17%) | |
| | | atrophic; 1 | |
| | | (17%) | |
| | | unknown | |
| Deceased at the time | 2 (40%; unrelated) | 3 (50%; | 3 (50%; 2 from |
| of last follow-up | | unrelated) | OSCC, 1 unrelated) |
| (cause, in relation to | | | |
| OSCC) | | | |
| OSCC Metastasis | N/A | N/A | 2 (33%) nodal |
| Dentures | 1 (20%) | 1 (17%) | 1 (17%) |
| OLP treatment, prior | N/A | 1 (17%; topical | 3 (50%; 2 topical |
| to biopsy | | corticosteroid) | corticosteroid; 1 |
| | | | topical tacrolimus) |
| Duration of disease | N/A | 45 (1-180) | 63 (6-120) |
| until biopsy, mean | | | |
| (months; range) | | | |

*For 1 sample of non-transforming OLP, p16 immunohistochemistry could not be performed due to insufficient available tissue.
HPV = human papillomavirus;
N/A = not applicable;
OSCC = oral squamous cell carcinoma;
SD = standard deviation.

TABLE 5
The most differentially over and under-expressed proteins in patients with
transforming OLP in relation to indolent OLP. Proteins with a log₂(fold change) of
≥25 or ≤−25 and false discovery rate <0.05 are shown. Protein descriptions were
obtained from StringDB or GeneCards (Szklarczyk et al., Nucl. Acids Res., 43
(Database issue): D447-452 (2015); and Stelzer et al., Curr. Protoc. Bioinformatics,
54: 1.30.31-1.30.33 (2016)).

| Gene | | Log2 Fold | Gaussian |
|---|---|---|---|
| Symbol | Protein function/relevance | Change | p value |
| TNNT3 | Troponin T is the tropomyosin-binding subunit of | 32.3 | 9.42E−07 |
| | troponin, the thin filament regulatory complex which | | |
| | confers calcium-sensitivity to striated muscle | | |
| | actomyosin ATPase activity and initiates the process of | | |
| | muscle contraction. | | |
| SYNM | Desmuslin or Synemin; a Type-VI intermediate filament | 31.6 | 1.06E−06 |
| | within the muscle cell cytoskeleton, that confer | | |
| | resistance to mechanical stress. It forms heteropolymeric | | |
| | intermediate filaments with desmin and/or vimentin, | | |
| | linking to adherens-type junctions within striated muscle | | |
| | cells. | | |
| MYH4 | Myosin-4 is located in myofibril and involved in muscle | 31.3 | 7.98E−07 |
| | contraction. | | |
| ST13P4 | Hsp70 Interacting Protein Pseudogene 4, predicted to | 31.2 | 8.88E−07 |
| | enable heat shock protein binding activity, chaperone | | |
| | cofactor-dependent protein refolding and protein- | | |
| | containing complex assembly. | | |
| MB | Myoglobin, serves as a reserve supply of oxygen and | 31.0 | 1.24E−06 |
| | facilitates the movement of oxygen within muscles. | | |
| MYLPF | Myosin light chain or MYL11, a structural component | 30.7 | 1.01E−06 |
| | of fast skeletal muscle. | | |
| CA1 | Carbonic anhydrase 1, catalyzes the reversible hydration | 28.7 | 4.40E−11 |
| | of carbon dioxide. They participate in a variety of | | |
| | biological processes, including saliva formation. | | |
| TNNC2 | Troponin C, central regulatory protein of striated muscle | 26.2 | 0.000128 |
| | contraction, which binds calcium and overcomes the | | |
| | inhibitory action of the troponin complex on actin | | |
| | filaments. | | |
| MYH1 | Myosin heavy chain 1, contractile protein which | 26.2 | 3.67E−05 |
| | converts chemical energy into mechanical energy | | |
| | through the hydrolysis of ATP. | | |
| SRPRB | Signal recognition particle receptor subunit beta, ensures | 25.3 | 5.71E−08 |
| | the correct targeting of the nascent secretory proteins to | | |
| | the endoplasmic reticulum membrane system. | | |
| FBLN1 | Fibulin-1, incorporated into fibronectin-containing | −27.8 | 4.71E−08 |
| | matrix fibers. May play a role in cell adhesion and | | |
| | migration within the extracellular matrix and its | | |
| | architecture especially that of the basement membranes. | | |
| | Has been implicated in a role in cellular transformation | | |
| | and tumor invasion and appears to be a tumor | | |
| | suppressor. | | |
| VCAN | Versican core protein, major component of the | −30.3 | 9.06E−57 |
| | extracellular matrix. Involved in cell adhesion and may | | |
| | play a role in intercellular signaling, cell proliferation, | | |
| | regulation of cell motility, migration and angiogenesis. | | |

TABLE 6
The most differentially over- and under-expressed proteins in patients with
indolent OLP and transforming OLP in relation to NOM. Proteins with a
log₂(fold change) of >25 or ≤−25 and false discovery rate <0.05 are shown.
Protein descriptions were obtained from StringDB or GeneCards
(Szklarczyk et al., supra; and Stelzer et al., supra)

| Gene | | Log2 Fold | Gaussian |
|---|---|---|---|
| Symbol | Protein function/relevance | Change | p value |
| LYZ | Lysozyme C, lysozymes have primarily a | 32.5 | 6.9E−107 |
| | bacteriolytic function. | | |
| TAPBP | Tap binding protein (tapasin), mediates interaction | 31.9 | 0 |
| | between newly assembled major histocompatibility | | |
| | complex class I molecules and the transporter | | |
| | associated with antigen processing, which is required | | |
| | for the transport of antigenic peptides across the | | |
| | endoplasmic reticulum membrane; essential for | | |
| | optimal peptide loading on the MHC class I | | |
| | molecule. | | |
| ANXA6 | Annexin A6, may associate with CD21, may regulate | 31.5 | 0 |
| | the release of Ca(2+) from intracellular stores; | | |
| ICAM1 | Intercellular adhesion molecule 1, ligands for the | 30.1 | 1.3E−83 |
| | leukocyte adhesion protein LFA-1 (integrin alpha- | | |
| | L/beta-2) and is typically expressed on endothelial | | |
| | cells and cells of the immune system. | | |
| CTSZ | Cathepsin Z, a lysosomal cysteine proteinase, also | 29.7 | 9.3E−09 |
| | been known as cathepsin X and cathepsin P. It is | | |
| | expressed ubiquitously in cancer cell lines and | | |
| | primary tumors and may be involved in | | |
| | tumorigenesis. | | |
| LBR | Lamin-B receptor; Anchors the lamina and the | 29.2 | 3.0E−126 |
| | heterochromatin to the inner nuclear membrane. | | |
| WARS | Tryptophanyl-TRNA Synthetase 1, catalyze the | 28.9 | 4.1E−13 |
| | aminoacylation of tRNA by their cognate amino acid. | | |
| | Regulates ERK, Akt, and eNOS activation pathways | | |
| | that are associated with angiogenesis, cytoskeletal | | |
| | reorganization and shear stress-responsive gene | | |
| | expression. | | |
| PTPRCAP | Protein Tyrosine Phosphatase Receptor Type C | 28.3 | 2.7E−125 |
| | Associated Protein, a transmembrane phosphoprotein | | |
| | specifically associated with tyrosine phosphatase | | |
| | PTPRC/CD45, a key regulator of T- and B- | | |
| | lymphocyte activation. | | |
| GBP1 | Guanylate-binding protein 1, hydrolyzes GTP to | 28.2 | 1.8E−12 |
| | GMP in 2 consecutive cleavage reactions. Exhibits | | |
| | antiviral activity against influenza virus, promote | | |
| | oxidative killing and deliver antimicrobial peptides to | | |
| | autophagolysosomes, providing broad host protection | | |
| | against different pathogen classes. | | |
| ERP29 | Endoplasmic reticulum resident protein 29, role in | 28.0 | 9.3E−09 |
| | the processing of secretory proteins within the | | |
| | endoplasmic reticulum. | | |
| PTPRC | Protein Tyrosine Phosphatase Receptor Type C, an | 27.9 | 1.3E−38 |
| | essential regulator of T- and B-cell antigen receptor | | |
| | signaling. This PTP also suppresses JAK kinases, | | |
| | and thus functions as a regulator of cytokine receptor | | |
| | signaling. | | |
| SOD2 | Superoxide dimutase, a mitochondrial protein that | 27.4 | 2.6E−07 |
| | binds to the superoxide byproducts of oxidative | | |
| | phosphorylation and converts them to hydrogen | | |
| | peroxide and diatomic oxygen. Mutations in this | | |
| | gene have been associated with idiopathic | | |
| | cardiomyopathy, premature aging, sporadic motor | | |
| | neuron disease, and cancer. | | |
| MAP7D1 | Predicted to be involved in microtubule cytoskeleton | 27.0 | 7.8E−08 |
| | organization. | | |
| HLA-DRA | HLA-DRA is one of the HLA class II alpha chain | 26.8 | 3.9E−13 |
| | paralogues, expressed on the surface of various | | |
| | antigen presenting cells such as B lymphocytes, | | |
| | dendritic cells, and monocytes/macrophages, and | | |
| | plays a central role in the immune system and | | |
| | response by presenting peptides derived from | | |
| | extracellular proteins, in particular, pathogen-derived | | |
| | peptides to T cells. | | |
| HMGB2 | High Mobility Group Box 2, a member of the non- | 26.5 | 4.4E−10 |
| | histone chromosomal high mobility group protein | | |
| | family; able to efficiently bend DNA and form DNA | | |
| | circles. Reported to be involved in the final ligation | | |
| | step in DNA end-joining processes of DNA double- | | |
| | strand breaks repair and V(D)J recombination. | | |
| EVL | Enah/Vasp-like, actin-associated proteins involved in | 26.0 | 5.9E−07 |
| | a range of processes dependent on cytoskeleton | | |
| | remodeling and cell polarity such as axon guidance | | |
| | and lamellipodial and filopodial dynamics in | | |
| | migrating cells. | | |
| KCTD12 | Potassium channel tetramerization domain containing | 25.7 | 5.4E−30 |
| | 12, enables identical protein binding activity. | | |
| | Predicted to be involved in protein homo- | | |
| | oligomerization. Predicted to act upstream of or | | |
| | within regulation of G protein-coupled receptor | | |
| | signaling pathway. | | |
| RHOG | Ras Homolog Family Member G, promote | 25.6 | 0.0003 |
| | reorganization of the actin cytoskeleton and regulate | | |
| | cell shape, attachment, and motility. | | |
| A2ML1 | Alpha-2-macroglobulin-like protein 1, acts as an | −27.7 | 0.0008 |
| | inhibitor of several proteases, form covalent | | |
| | interactions with proteases, and has been reported as | | |
| | the p170 antigen recognized by autoantibodies in the | | |
| | autoimmune disease paraneoplastic pemphigus. | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-18. (canceled)

19. A method for treating a mammal having OLP, wherein said method comprises:
(a) determining that a cell of said OLP comprises a mutant version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of nucleic acid encoding a TENM3 polypeptide, a mutated version of nucleic acid encoding an ASH1L polypeptide, a mutated version of nucleic acid encoding an OBSCN polypeptide, a mutated version of nucleic acid encoding a TTRAP polypeptide, or a mutated version of nucleic acid encoding a LRP2 polypeptide, thereby identifying said OLP as being likely to progress to OSCC, and
(b) administering to said mammal an immunosuppressive or immunomodulatory agent.

20. The method of claim 19, wherein the mammal is a human.

21. The method of claim 19, wherein said cell is within a cytology brushing sample.

22. The method of claim 19, wherein said cell is a squamous epithelial cell or a stromal cell affected by said OLP.

23-31. (canceled)

32. The method of claim 19, wherein said method comprises determining that said cell comprises said mutant version of nucleic acid encoding a TP53 polypeptide, said mutated version of nucleic acid encoding a CELSR1 polypeptide, said mutated version of nucleic acid encoding a CASP8 polypeptide, said mutated version of nucleic acid encoding a KMT2D polypeptide, said mutated version of nucleic acid encoding a TENM3 polypeptide, said mutated version of nucleic acid encoding an ASH1L polypeptide, said mutated version of nucleic acid encoding an OBSCN polypeptide, said mutated version of nucleic acid encoding a TTRAP polypeptide, and said mutated version of nucleic acid encoding a LRP2 polypeptide.

33. The method of claim 19, wherein said immunosuppressive or immunomodulatory agent comprises a corticosteroid, dapsone, azathioprine, tacrolimus, mycophenolate mofetil, or a biologic agent.

34. A method for treating OLP, wherein said method comprises administering, to a mammal identified as having a cell of said OLP comprising a mutant version of nucleic acid encoding a TP53 polypeptide, a mutated version of nucleic acid encoding a CELSR1 polypeptide, a mutated version of nucleic acid encoding a CASP8 polypeptide, a mutated version of nucleic acid encoding a KMT2D polypeptide, a mutated version of nucleic acid encoding a TENM3 polypeptide, a mutated version of nucleic acid encoding an ASH1L polypeptide, a mutated version of nucleic acid encoding an OBSCN polypeptide, a mutated version of nucleic acid encoding a TTRAP polypeptide, or a mutated version of nucleic acid encoding a LRP2 polypeptide, an immunosuppressive or immunomodulatory agent.

35. The method of claim 34, wherein the mammal is a human.

36. The method of claim 34, wherein said cell is a squamous epithelial cell or a stromal cell affected by said OLP.

37-45. (canceled)

46. The method of claim 34, wherein said mammal is a mammal identified as having as cell of said OLP comprising said mutant version of nucleic acid encoding a TP53 polypeptide, said mutated version of nucleic acid encoding a CELSR1 polypeptide, said mutated version of nucleic acid encoding a CASP8 polypeptide, said mutated version of nucleic acid encoding a KMT2D polypeptide, said mutated version of nucleic acid encoding a TENM3 polypeptide, said mutated version of nucleic acid encoding an ASH1L polypeptide, said mutated version of nucleic acid encoding an OBSCN polypeptide, said mutated version of nucleic acid encoding a TTRAP polypeptide, and said mutated version of nucleic acid encoding a LRP2 polypeptide.

47. The method of claim 34, wherein said immunosuppressive or immunomodulatory agent comprises a corticosteroid, dapsone, azathioprine, tacrolimus, mycophenolate mofetil, or a biologic agent.

48-86. (canceled)

87. A method for treating OLP, wherein said method comprises administering, to a mammal identified as having a cell of said OLP comprising elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB, an immunosuppressive or immunomodulatory agent.

88. The method of claim 87, wherein the mammal is a human.

89. The method of 87, wherein said cell is a squamous epithelial cell or a stromal cell affected by said OLP.

90-99. (canceled)

100. The method of claim 87, wherein said mammal is a mammal identified as having as cell of said OLP comprising said elevated expression of CA1, said elevated expression of TNNT3, said elevated expression of SYNM, said elevated expression of MB, said elevated expression of MYH4, said elevated expression of ST13P4, said elevated expression of MYLPF, said elevated expression of MYH1, said elevated expression of TNNC2, and said elevated expression of SRPRB.

101. The method of claim 87, wherein said elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB comprises elevated expression of a CA1 mRNA, elevated expression of a TNNT3 mRNA, elevated expression of a SYNM mRNA, elevated expression of a MB mRNA, elevated expression of a MYH4 mRNA, elevated expression of a ST13P4 mRNA, elevated expression of a MYLPF mRNA, elevated expression of a MYH1 mRNA, elevated expression of a TNNC2 mRNA, elevated expression of a SRPRB mRNA, or a combination thereof.

102. The method of claim 87, wherein said elevated expression of CA1, elevated expression of TNNT3, elevated expression of SYNM, elevated expression of MB, elevated expression of MYH4, elevated expression of ST13P4, elevated expression of MYLPF, elevated expression of MYH1, elevated expression of TNNC2, or elevated expression of SRPRB comprises elevated expression of a CA1 polypeptide, elevated expression of a TNNT3 polypeptide, elevated expression of a SYNM polypeptide, elevated expression of a MB polypeptide, elevated expression of a MYH4 polypeptide, elevated expression of a MYLPF polypeptide, elevated expression of a MYH1 polypeptide, elevated expression of a TNNC2 polypeptide, elevated expression of a SRPRB polypeptide, or a combination thereof.

103. The method of claim 87, wherein said immunosuppressive or immunomodulatory agent comprises a corticosteroid, dapsone, azathioprine, tacrolimus, mycophenolate mofetil, or a biologic agent.
